(19) United States
(12) Patent Application Publication
(10) Pub. No.: US 2025/0359822 A1
(43) Pub. Date: Nov. 27, 2025

(54) Non-Destructive Pressure-Assisted Tissue Stiffness Measurement Apparatus

(71) Applicants: The Trustees of the Stevens Institute of Technology, Hoboken, NJ (US); Vanderbilt University, Nashville, TN (US); The Trustees of Columbia University in the City of New York, New York, NY (US); The Board of Trustees of the Leland Stanford Junior University, Stanford, CA (US)

(72) Inventors: Jinho Kim, Jersey City, NJ (US); Gordana Vunjak-Novakovic, New York, NY (US); John D. O'Neill, New York, NY (US); Meghan Pinezich, New York, NY (US); Brandon A. Guenthart, Stanford, CA (US); Seyed Mohammad Mir, Hoboken, NJ (US); Jiawen Chen, Hoboken, NJ (US); Matthew Bacchetta, Nashville, TN (US)

(73) Assignees: The Trustees of the Stevens Institute of Technology, Hoboken, NJ (US); Vanderbilt University, Nashville, TN (US); The Trustees of Columbia University in the City of New York, New York, NY (US); The Board of Trustees of the Leland Stanford Junior University, Stanford, CA (US)

(21) Appl. No.: 19/088,683

(22) Filed: Mar. 24, 2025

Related U.S. Application Data
(63) Continuation-in-part of application No. 18/696,898, filed on Mar. 28, 2024.
(60) Provisional application No. 63/250,123, filed on Sep. 29, 2021.
(60) Provisional application No. 63/569,664, filed on Mar. 25, 2024.

(51) Int. Cl. A61B 5/00; A61B 1/07; A61B 5/0538; A61B 5/08; A61B 8/00; A61B 8/12; A61M 25/01

(52) U.S. Cl. CPC A61B 5/6852; A61B 1/07; A61B 5/0053; A61B 5/0071; A61B 5/0084; A61B 5/0538; A61B 5/08; A61B 8/12; A61B 8/485; A61B 2562/0247; A61M 25/0113

(57) ABSTRACT

A minimally invasive device, containing a pressure channel, camera, and optical fiber imaging probe, to measure the stiffness of tissues in vivo and ex vivo is disclosed. To measure tissue stiffness in vivo, the device is inserted into a patient and navigated to a tissue of interest, where stiffness is evaluated by applying suction and measuring the elongation or by applying compression force and measuring the compression of the tissue. Biopsies can be taken for further analysis, or tissue can be removed using an ablation laser. Small fluorescent molecules or therapeutics can also be delivered for improved visualization and targeted treatment. As such, this technology may be used to evaluate the stiffness of biomaterials as well as tissues and organs that are difficult to access, allowing for simultaneous diagnosis, treatment, and excision of diseased tissues.

### CROSS-REFERENCE TO RELATED APPLICATIONS

The present application is a continuation-in-part of U.S. patent application Ser. No. 18/696,898, filed Mar. 28, 2024, which is an application under 35 U.S.C. § 371 of International Application No. PCT/US2022/077311, filed Sep. 29, 2022, which claims priority to U.S. Provisional Patent Application 63/250,123 filed Sep. 29, 2021, all of which being incorporated by reference herein in their entirety. This application also claims priority to U.S. Provisional Patent Application 63/569,664, filed Mar. 25, 2024, which is also incorporated by reference herein in its entirety.

### STATEMENT REGARDING FEDERALLY SPONSORED RESEARCH

This invention was made with government support under P41 EB027062 awarded by the National Institutes of Health and 2143620 awarded by the National Science Foundation. The government has certain rights in the invention.

### FIELD OF THE INVENTION

The present invention relates to the measurement of mechanical properties, and, specifically, to the determination of elastic modulus of soft tissues, organs, and biomaterials without compromising their native structure.

### BACKGROUND OF THE INVENTION

In biological tissues, mechanical stiffness plays a fundamental role in cell and tissue function. Alterations in the stiffness, or elasticity, of tissues can induce pathological interactions that affect cellular activity and tissue function. Stiffness refers to the resistance of tissue to deformation in response to an applied force, and it is often represented by elastic modulus (E). Several studies have revealed that tissue development and remodeling are regulated not only by biochemical regulators but also by biophysical cues. Specifically, alterations in tissue stiffness strongly correlate with and contribute to many diseases and pathologies, such as tissue fibrosis, cancer, sclerosis, and atherosclerosis. For instance, fibrotic tissues are stiffer than normal tissues due to increased extracellular matrix (ECM) synthesis and deposition during tissue remodeling. Similarly, tumors in various cancers (e.g., lung, breast, and liver cancers) show greater stiffness than surrounding healthy tissues due to changes in components of cells and ECM, as well as disruption of interstitial fluid balance in tumors. Hence, stiffness assessment can be utilized as a diagnostic tool for understanding the underlying diseases and pathologies and making disease-specific interventions The integral connection between tissue stiffness and disease highlights the importance of accurate quantitative characterizations of soft tissue mechanics, which can improve understanding of disease and inform therapeutic development. For example, accurate evaluation of the mechanical properties of lung tissue has been especially challenging due to its anatomical and mechanobiological complexities. Discrepancies in the measured mechanical properties of dissected lung tissue samples and intact lung tissue in vivo have limited the ability to accurately characterize intrinsic lung mechanics.

Current devices and methods for measuring the stiffness of soft tissues are limited to the surface of accessible tissues and require the operator to rely on their vision to place the devices. This limits the ability of researchers and surgeons to understand both healthy and diseased tissue properties, which understanding is critical in advancing diagnostics and treatments of soft tissue diseases.

Robot-Assisted minimally invasive surgery (RMIS) has emerged as an approach that allows surgeons to perform complicated surgical procedures with improved dexterity, visualization, and precision and accuracy that can collectively enhance treatment outcomes. Advanced robotic surgical systems, such as the da Vinci® system (Intuitive Surgical, Inc.) and Senhance Surgical System (TransEntrix Inc.), offer multiple advantages, including increased degrees of freedom, high-definition visualization of the surgical site with accurate depth perception, and enhanced scalability. RMIS performed using these surgical systems provides unique benefits to patients, including reduced pain and discomfort, smaller incisions, minimal blood loss, and faster recovery time. Accordingly, RMIS is becoming increasingly used for a wide range of specialties, including thoracoscopic, hepatobiliary, gynecologic, urologic and gastrointestinal surgery.

Despite the numerous advantages and benefits, one of the widely recognized limitations of RMIS is the absence of tactile sensations (i.e., touch-and force-related sensations). During traditional open surgery, surgeons often use tactile feedback through manual palpation to examine the pathologic conditions of the tissues. In particular, because pathologic tissues, such as tumors and fibrosis, are stiffer than normal tissues, intra-operative manual palpation enables surgeons to identify diseased tissues that must be surgically treated. However, during RMIS, surgeons rely on visual information to assess the tissues because the use of robot arms for surgical operation limits their ability to receive tactile feedback. Pre-operative imaging-based analysis modalities, such as computed tomography (CT), magnetic resonance imaging (MRI), and elastography remain limited by their special resolution and only provide a single historical snapshot which is often difficult for the surgeon to utilize in real-time during an operation.

### SUMMARY OF THE INVENTION

The present invention involves a device that can measure stiffness of a wide variety of tissues, organs, and biomaterials in a non-destructive and rapid manner, as well as methods of using such a device to quantify tissue or material stiffness (FIGS. 1A-1B, 2C; 28A-2B, 31A). The inventive device is a pressure-assisted device that is able to evaluate the stiffness of biomaterials, tissues, and organs in a non-destructive and/or minimally invasive manner, thereby allowing accurate and rapid quantification of tissue and organ stiffness in vivo and ex vivo. Furthermore, the device can be applied to detect, treat, and/or remove the injured or diseased tissue.

Conventional methods, such as tensile and compression tests, require isolation of tissue samples for the measurements that can result in substantial alteration in native tissue structure and anatomy, leading to inaccurate readouts. The inventive device allows for vacuum or compression-assisted direct in situ measurement of local tissue without the need of tissue sampling, allowing for evaluation of tissues and organs that are difficult to access. The device can be designed with a steerable and conformable configuration such that it can be inserted and placed locally into the measurement sites within the patient's body that are difficult access, such as the respiratory, gastrointestinal, and urinary tracts.

The inventive device is integrated with a miniaturized camera or optical fiber imaging probe that allows clinicians to accurately determine the position of the device during its insertion and navigation within the patient's body, thereby facilitating placement of the device to target locations with improved spatial resolution for stiffness measurements (FIGS. 12A-12C, 13A-13C, 30A-30E, 36A-36B, 37A-37B). Furthermore, the measurement device can be conformable, steerable, thin (e.g., diameter less than 5 mm), and long (e.g., length of approximately 1 m), allowing minimally invasive device insertion via a small incision opening created in the patient's body and placement of the device onto any tissue or organ surface for measurements, such as lung, respiratory tract, liver, heart, brain, or intestines.

In addition, if the inventive device is configured as a balloon-integrated probe, measurement of internal tissue stiffness can be achieved (FIG. 4). When equipped with a balloon, the inventive probe can be introduced locally into the lung tissue via a syringe needle, wherein the balloon can be easily expanded inside the lung tissue by introducing air or fluid. Pressure and volume inside the balloon can be determined in real time via a pressure sensor and a volume sensor, respectively, that are connected to a pump externally, allowing accurate quantification of tissue stiffness.

The present invention contains many possible commercial applications: The inventive probe can alleviate the major challenges encountered during tumor resection surgery that arise due to difficulty identifying the boundaries of the tumor, so that it can be ensured that the entire tumor is removed during surgery. Specifically, the inventive device can serve as an intraoperative tool to determine the margins of a tumor in real-time to facilitate complete removal of tumors. Additionally, there are applications in mechanical testing to evaluate injury and function in donor organs to determine suitability for transplantation, including during ex vivo lung perfusion. Another potential use is detection of, targeted delivery to, and removal of injured or diseased tissue from various organs (e.g., gut polyps, lung fibrotic foci, etc.). The present invention may also be used for characterization of the mechanical properties of other organs and tissues, including gut, skin, vasculature, liver, etc. for research, diagnostic, prognostic, and therapeutic purposes. Mechanical evaluation of stem cell-tissue and cell-cell binding interactions is also enabled. A not-necessarily-final example of use is diagnosis and treatment of atherosclerosis. The device of the present invention can be used to measure the artery stiffness for patients prone to atherosclerosis and to remove the built-up fat, cholesterol, or calcium. Veterinary applications are also possible.

A method in accordance with the present invention can involve stiffness measurement of a tissue of interest that entails providing a probe having a compression head; locating the probe such that the compression head is proximate the tissue of interest; applying a pressure to the compression head; detecting a response at the tissue of interest in response to the pressure applied via the applying step; and calculating one or more physical properties of the tissue of interest based on the response (FIGS. 23A-23B, 25A-25B, 26, 27A-27B, 28C-28D). The method can be performed on the tissue of interest in in vivo conditions, in which case the probe is inserted into a patient, or in ex vivo conditions. For in vivo applications, the tissue of interest can be imaged using an imaging element, which can be, for example, an optical fiber probe or a miniaturized camera. Additionally, ablation of damaged or otherwise problematic tissue can be performed with a laser localized on the probe. Furthermore, therapeutic compounds and/or fluorescent molecules can be delivered simultaneously to the tissue of interest.

In one embodiment, the probe is introduced via a syringe needle proximate the tissue of interest (FIG. 4). The probe can also be a balloon probe capable of being inflated to monitor its pressure and volume at the tissue of interest.

The method can also entail regulation of the pressure applied to the compression head (e.g., via a controller) (FIGS. 23A-23B, 31C-31D). In another embodiment, the calculation step involves determining tissue stiffness (FIGS. 27A-27B, 31A). In another embodiment, the tissue of interest can be a tumor, whose boundaries can be determined in real-time (e.g., via computer vision) (FIGS. 32A-32C, 33C-33D).

In another embodiment, contact electrodes are placed proximate the tissue of interest, and the maximum tissue deformation is determined. Upon contact, these electrodes can also measure the electrical resistance of the tissue of interest (FIGS. 23A-23B, 28C-28D, 31B). In yet another embodiment, the pressure is applied as suction force, and elongation length of the tissue of interest in response to the suction force is measured (FIGS. 2A-2C, 3A-3B). In a still further embodiment, the pressure is applied as compressive force, and tissue deformation length of the tissue of interest in response to the compressive force is measured (FIGS. 28D, 31B). Both tissues or synthetic biomaterials can be evaluated using such methods.

In another embodiment of the present invention, a device for evaluating stiffness of materials can be provided (FIGS. 28A-28B). The device can include a compression head; an imaging element coupled to the compression head; a motorized steering means adapted to move the imaging element and the compression head; a pressure network (e.g., a pressure line) adapted to apply positive or negative pressure to the compression head; and a controller adapted to regulate and control the pressure network.

In one embodiment, the pressure line and imaging element are integrated with the motorized steering means as part of a steerable compartment of the device (FIGS. 14A-14B, 15A-15C, 16A-16C, 30A-30C). The imaging element can be an optical fiber probe or a miniaturized camera. In one embodiment, the controller is adapted to regulate pressure applied to the compression head, analyze collected tissue deformation data and calculate tissue stiffness (FIGS. 27B, 31A). The inventive device can also include ablation means (e.g., a laser). In another embodiment, the device also includes a delivery means for delivering therapeutic compounds to a tissue of interest (FIG. 5A). The delivery means can be further adapted to deliver fluorescent molecules. In one embodiment, the device has a diameter less than 5 mm and a length of at least one meter.

In a further embodiment, the inventive device has a balloon probe, adapted to be introduced via a syringe needle, wherein the balloon probe can be expanded to monitor pressure and volume at a tissue of interest (FIG. 4). In certain applications, the inventive device can be adapted for use as an intraoperative tool to determine tumor boundaries in real-time. For instance, the device can utilize computer vision to analyze the tissue of interest. The device can be adapted to determine elongation length of the tissue of interest (FIGS. 2A-2B, 3A-3B, FIG. 4), the length of tissue deformation under compression in the tissue of interest (FIGS. 23A, 28D), or the electrical resistance of the tissue of interest (FIGS. 28D, 31B).

In additional embodiments, the compression head is a dome-shaped tip. The compression head can further include contact electrodes and a force sensor that monitor the compression force applied to the tissue of interest (FIGS. 23A-23B, 28C-28D).

It is an object of the present invention to provide a minimally invasive probe capable of rapid and accurate quantification of tissue stiffness.

A second object of the present invention is to provide a probe that contains a motorized steerable compartment for minimally invasive insertion into the body.

It is another object of the present invention to provide a probe that is capable of applying a pressure network capable of providing negative or positive pressure to the tissue of interest.

It is yet another object of the present invention to provide an optical fiber probe that utilizes a miniaturized camera for guiding the navigation of the device and monitoring tissue deformation

It is a further object of the present invention to provide a device that incorporates a computer-based controller that regulates the pressure, analyzes collected tissue deformation data, and calculates tissue stiffness, and profile two-dimensional (2D) stiffness map.

It is an additional object of the present invention to provide a device that integrates the pressure line, imaging probes, and camera into the steerable compartment of the device.

It is yet another object of the present invention to provide a probe that enables stiffness measurements of internal tissues via an inflatable balloon needle.

It is another object of the present invention to provide a probe capable of (i) introducing fluorescent molecules to a target region for enhanced imaging and/or (ii) locally delivering therapeutics to the tissue of interest.

It is a not-necessarily final object of the present invention to provide a probe that enables the removal of tissue by laser ablation or biopsy.

In yet another embodiment of the present invention, a device that can pinpoint the location of diseased tissues with altered stiffness during robot-assisted surgery disclosed. The device includes a device that can measure the stiffness of a wide variety of tissues, organs, and biomaterials in a rapid and non-invasive manner (FIGS. 28A-28D), which includes three sub-components: 1) a deployable, highly sensitive probe mounted on a steerable catheter that can compress local tissue for stiffness measurement (FIGS. 28A-28D); 2) a motion control module that enables multi-directional device movements, such as linear displacement, rotation, and deflection of the device (FIGS. 29A-29C 29, 35A-35H, 36A-36B); and 3) a micro-optical imaging module that provides visual information during the probe navigation and tissue stiffness measurement (FIGS. 30A-D, 36A-36B, 37A-37B, 43A-43C). The present invention also pertains to a method of use of this device to quantify the stiffness of tissues and profile the stiffness map (FIGS. 23A-23B, 24, 25A-25B, 26, 27A-27B, 31A-31F, 32A-32C, 33A-33C, 38A-38B).

In such embodiments, a robotic tissue palpation device that can evaluate the stiffness of tissues and organs in a non-destructive and/or minimally invasive manner would be achieved, thereby allowing accurate and rapid quantification of tissue and organ stiffness in vivo and ex vivo. Such a device can be applied to detect, treat, and/or remove injured or diseased tissues, such as tumors and fibrosis, during robotic surgery.

Such a modified device can be integrated with a sensing probe, steerable catheter, a motion control module, and an optical fiber-based imaging module. The combination of these features would enable surgeons to accurately identify and differentiate between healthy and diseased tissues with improved precision and efficiency, during robot-assisted surgeries.

An object of the inventive modified device is achieving non-destructive in situ measurement. Unlike conventional methods that require tissue isolation and can potentially alter the tissue structure, the modified robotic tissue palpation device enables direct measurement of local tissue stiffness without the need for tissue sampling. This non-destructive approach ensures accurate results while preserving the native tissue structure and anatomy.

Another object of the inventive modified device is to provide a sensitive measurement probe. The sensing probe allows fast and accurate monitoring of pressure applied on tissue during stiffness measurements. The probe can be equipped with highly sensitive force sensor that detects the magnitude of force exerted on the tissue, ensuring reliable and consistent stiffness measurements.

A further object of the inventive modified device is enabling a novel method for informing the endpoints of measurements. One innovative feature of such a device is the integrated contact electrodes circuit to inform the maximum tissue deformation. Deformation of tissue to a specified degree can be accurately determined through non-invasive, real-time recording of voltage via the electrodes. Current deformation measurement methods, such as optically based and computer-assisted approaches, are time-consuming and prone to errors. On the other hand, predetermined magnitudes of tissue deformation can be achieved intraoperatively using the present approach. Further, the deformation length can be easily customized by using a hemispheric indenter with different heights. The magnitudes of electrical voltage and current (voltage: 3.2 volts, current: 0.5 mA) could also be easily adjusted to different values to be within a safe and clinically relevant range.

Yet another object of the inventive modified device is facilitating accessibility to challenging measurement sites. Designed with steerable and conformable features, the present device can be inserted and positioned within difficult-to-access measurement sites in the patient's body during minimally invasive surgery. This includes areas such as the respiratory, gastrointestinal, and urinary tracts, allowing for comprehensive evaluation of tissues and organs that were previously hard to reach.

A further object of the inventive modified device is enabling accurate tumor and fibrosis detection. The inventive device has been designed to provide a rapid and precise solution for detecting diseased tissues with altered stiffness, such as tumors and fibrosis, during robotic surgery. By evaluating the stiffness of suspicious tissues and profiling tissue stiffness maps in real-time during robot-assisted procedures, surgeons can make objective and data-driven decisions for surgically removing diseased tissues.

Another object of the inventive modified device is enabling precise motion control. In the present embodiment of the robotic palpation device, multi-directional movements, including translational, tilting, and deflection motions, are enabled by simultaneously controlling motors. The conformable and controllable device motions facilitate device navigation and tissue compression within tight spaces, such as the chest cavity, during robotic surgery. In particular, the wire-driven design of the catheter can provide dexterity and manipulability, allowing the probe to apply the normal force to tissue with irregular surface topology. The majority of palpation devices reported in the literature have a limited range of motion and flexibility, which makes them difficult to use during robotic surgery. In contrast, the present device is capable of maneuvering in confined spaces, allowing surgeons to access the surgical targets and survey questionable tissue rapidly. Such a device may be able to be integrate into a standard robotic or laparoscopic device arm (port diameter: 5-12 mm) and be controlled by the surgeon with existing interfaces.

A not-necessarily final object of the inventive modified device is providing a new imaging module and image processing algorithms. The optical fiber imaging probe incorporated into the device allows visual monitoring of the local tissue during stiffness measurement. Notably, the imaging module can be customized to enable visualization at the cellular level. By implementing a real-time image processing scheme, such as Gaussian filtering, the quality of images and videos can be substantially improved. In addition, the flexibility of the imaging fiber facilitates its integration into the steerable catheter. Further, the bifurcated geometry of the imaging fiber enables simultaneous illumination and imaging, with the light passing through the “transmitting bundle” to the fiber tip and the fluorescent signal passing through the “receiving bundle” into the camera. This imaging capability can be useful during intra-operative tumor resection, where surgeons can administer fluorescent molecules that can specifically label tumors to improve the accuracy of tumor identification and resection.

In summary, the modified inventive device allows for multiple degrees of freedom. Additionally, the modified inventive device can not only detect tumors but also other diseased tissues with altered stiffness, such as fibrotic tissues. Further features include an improved fiber optic imaging module, new steerable catheter movements (translational, tilting, and deflection), an improved sensing probe, new image processing algorithms, new experimental models with animal tissue phantoms, a contact electrode arrangement for informing the endpoint of stiffness measurement (i.e., maximum deformation), a force sensor circuit, and a thin film based force sensor.

The main application of such a robotic tissue palpation device is identifying the exact boundaries of tumors during robotic surgery. Frequently, during robotic surgery, surgeons find it challenging to pinpoint the margins of tumor tissues, in particular, deep-seated tumors smaller than 1 cm. The present device can serve as an intraoperative tissue assessment tool to determine the margins of tumors, to facilitate the surgical removal of these diseased tissues. Future applications could expand beyond this main use, however.

For instance, the robotic palpation device can also be used for detection of fibrotic tissues. Fibrotic tissues are stiffer than normal tissues due to the excessive accumulation of extracellular matrix components, such as collagen and other fibrous proteins. By determining the stiffness of the tissue, the devices can differentiate fibrotic and normal tissues.

Additionally, the tissue palpation device can be used as a diagnostic tool to evaluate the health of rejected donor lungs recovering in ex vivo lung perfusion (EVLP) and cross-circulation platforms.

Further, the device can measure the mechanical properties of other organs and tissues, including gut, skin, vasculature, liver, etc. for research, diagnostic, prognostic, and therapeutic purposes. In other applications, the device can be used for other diseased tissues with altered

stiffness. For example, it can be used to measure artery stiffness for patients that are prone to measuring of mechanical properties of other organs and tissues, including gut, skin, vasculature, liver, etc., for research, diagnostic, prognostic, and therapeutic purposes.

In another application, the device can be used in sports medicine. Through measuring muscle stiffness, the device is capable of monitoring muscle recovery after injury or exercise. For instance, damaged muscle is stiffer than normal muscle, and as the muscle heals, its stiffness decreases. The device can monitor muscle recovery after injury or exercise to determine when the muscle is ready to resume full activity. Additionally, the device can be used to optimize training regiments. Stiff muscles are susceptible to injury. Monitoring stiffness therefore enables sports medicine practitioners to design muscle-friendly training regiments to maximize training effectiveness while preventing injuries.

Moreover, the device can be utilized as a hand-held device for cosmetic purposes. The device can evaluate the quality of skin tissue and provide skin treatments. With aging, skin naturally loses elasticity, which is directly related to skin stiffness. In addition, eczematous skin, characterized by dryness and inflammation, is stiffer and less pliable than normal skin. Therefore, the device can assess the elasticity of the targeted areas and provide treatments, such as physical stimulation, cosmetics application, laser therapy, and electrical stimulation.

Further, the device may also have potential applications in the agricultural field, specifically in assessing the quality of ripe fruits during harvest. The palpation device can be utilized in a robot to effectively determine the stiffness of fruits, which varies between unripe and ripe states. This enables robots to determine unripe and ripe fruit during the harvesting process.

A not-necessarily-final application of the imaging system developed is use during intra-operative tumor resection procedures, where surgeons can administer fluorescent molecules that can specifically label tumors to improve the accuracy of tumor identification and resection.

### BRIEF DESCRIPTION OF THE DRAWINGS

For a more complete understanding of the present invention, reference is made to the following detailed description of various exemplary embodiments considered in conjunction with the accompanying drawings, in which:

FIG. 1A is a schematic of an apparatus to measure local tissue stiffness;

FIG. 1B is a schematic showing a probe to measure local tissue stiffness;

FIG. 2A is a schematic illustration of insertion of the apparatus of FIGS. 1A-1B into a thoracic cavity;

FIG. 2B is a schematic illustration of video-based acquisition of elongation length data from the apparatus of FIG. 2A;

FIG. 2C is a table showing equations for calculating elastic modulus;

FIG. 3A is a schematic illustration of insertion of the apparatus of FIGS. 1A-1B into a respiratory tract;

FIG. 3B is a schematic illustration of video-based acquisition of elongation length data from the apparatus of FIG. 3A;

FIG. 4. is a schematic illustration showing measurement of tissue stiffness using a needle integrated with an expandable balloon in accordance with an embodiment of the present invention;

FIG. 5A is a schematic illustration showing localized removal of tissue via biochemical treatment in accordance with an embodiment of the present invention;

FIG. 5B is a schematic illustration showing localized removal of tissue via laser treatment in accordance with an embodiment of the present invention;

FIGS. 6A-D involve (i) an overview of vacuum-based measurement of elastic moduli of soft biomaterials, showing a hydrogel (FIG. 6A), (ii) lung tissue deformed via vacuum pressure (FIG. 6B), (iii) a custom-built system to measure the stiffness of soft biomaterials and soft biological tissues (FIG. 6C), and (iv) a schematic of the measurement system of FIG. 6C (FIG. 6D);

FIG. 7A is a series of images showing a hydrogel undergoing stiffness measurement in accordance with an embodiment of the present invention;

FIG. 7B is a merged image created from the images of FIG. 7A;

FIG. 8A is a series of graphs showing applied negative pressure and measured elongation length for various hydrogels undergoing cyclic vacuum-loading in accordance with an embodiment of the present invention;

FIG. 8B is a series of graphs showing measured elongation length for various hydrogels undergoing the cyclic vacuum-loading process of FIG. 8A in accordance with an embodiment of the present invention;

FIG. 8C is a series of graphs showing elastic moduli for the various hydrogels based on the measurements of FIGS. 8A and 8B;

FIG. 9A is a micrograph showing aerated gelatin;

FIG. 9B is a series of images showing the gelatin of FIG. 9A deformed under vacuum pressure;

FIG. 9C is a graph showing deformation of the aerated gelatin of FIG. 9A and a non-aerated control gelatin sample;

FIG. 9D is a graph showing elastic moduli for the control sample and aerated gelatin of FIG. 9C;

FIG. 10Ai is a photograph of a setup for measuring the stiffness of an ex vivo rat lung in accordance with an embodiment of the present invention;

FIG. 10Aii is a schematic of a setup for measuring the stiffness of an ex vivo rat lung in accordance with an embodiment of the present invention;

FIG. 10B is a series of images showing deformation under vacuum pressure of tissue from the lung of FIG. 10Ai-10Aii;

FIG. 10C is a graph showing maximum elongation length of the lung tissue of FIG. 10B;

FIG. 10D is a graph showing elastic modulus of the lung tissue of FIG. 10B for various pressure values;

FIG. 10E is a force diagram showing tension (T) generated across the pleural surface of the lung tissue of FIGS. 10Ai and 10Aii-10B due to increased P_{Alv} that leads to increased P_{V} required to stretch the lung tissue;

FIG. 11A is a schematic of lung injury induced by intratracheal instillation of trypsin with ICG fluorescent dye;

FIG. 11B is an image showing a photograph (i) and a NIR image (ii) of explanted rat lungs;

FIG. 11C is a graph showing pressure-volume curves of the rat lungs of FIG. 11B before and after injury that were obtained by measuring intra-alveolar pressure P_{Alv} and volume V_{L}; of air inspired or expired through the trachea of the lung using a small animal ventilator;

FIG. 11D is a series of H&E images of alveoli of the lungs of FIG. 11B, showing (i) control (i.e., healthy); and (ii) acute injured rat lungs

FIG. 11E is a graph illustrating maximum elongation length of the lungs of FIG. 11B;

FIG. 11F is a graph showing elastic moduli of the lungs of FIG. 11B under different P_{Alv} for both injured and control lungs;

FIG. 12A is photograph showing a custom-built imaging system for use with the optical imaging probe of the present invention;

FIG. 12B shows a front view imaging probe in accordance with an embodiment of the present invention (12B(i)) and bright-field imaging (12B(ii)) and fluorescent imaging (12B(iii)) achieved using the probe of FIG. 12B(i);

FIG. 12C shows a side view imaging probe in accordance with an embodiment of the present invention (12C(i)) along with fluorescent images obtained using the probe following injection of fluorescently labeled 10-um microparticles (12C(ii)) and fluorescently labeled mesenchymal stem cells (red) into the rat trachea (12C(iii));

FIG. 13A shows a photograph and a schematic diagram illustrating insertion of the front view imaging probe of FIG. 12B into a rat lung;

FIG. 13B is a pair of bright-field images obtained from the rat lung shown FIG. 13A;

FIG. 13C is a pair of fluorescent images obtained from the rat lung of FIG. 13A;

FIG. 14A is a 3D drawing of a motorized steerable catheter device constructed in accordance with an embodiment of the present invention (i), the steerable distal end (ii) of the device, and a schematic showing deflection of the distal end of the device achieved via servo motors and pulling wires integrated into the system (iii);

FIG. 14B is a photograph of a prototype of the motorized steerable catheter device of FIG. 14A;

FIGS. 15A-15C is a series of photographs illustrating vision-assisted tracking of a target using the prototype of FIG. 14B; and

FIGS. 16A-C involve a series of images showing insertion of the prototype device (see FIG. 14B) into the respiratory tract of explanted pig lung, including photographic images showing the process of device insertion into a plastic port connected to the trachea of the pig lung (FIG. 16A), a photograph of the prototype device placed into the pig lung airways (FIG. 16B), and a visualization of the airway interior using the prototype device inserted into the lung (FIG. 16C);

FIGS. 17A-17B constitute a series of diagrams illustrating the correlation between probe diameter and tissue elongation depth for a 0.58 mm probe (FIG. 17A) and a 1.5 mm probe (FIG. 17B);

FIG. 18 is a graph illustrating the effects of vacuum pressure on tissue elongation length;

FIG. 19 is a graph illustrating effects of rate change (S_{P}) of magnitude of the negative pressure (|P_{V}|) on tissue elongation length (L_{E});

FIG. 20 is a graph illustrating the correlation between vacuum pressure (|P_{V}|) and tissue elongation length (L_{E});

FIG. 21 is a force diagram showing tension force (T) within the tissue network and alveolar surface tension force (F_{ST}) at the air-liquid interface where increasing P_{Alv} results in elevated T and F_{ST} leading to greater P_{V} needed to stretch the lung parenchymal tissue;

FIGS. 22A-2B are a series of photographs illustrating lung pleura integrity for healthy (FIG. 22A) and trypsinized (FIG. 22B) rat lungs in accordance with an embodiment of the present invention;

FIGS. 23A-23B are a schematic representations of a compression-based tissue palpation device and its operation procedure in accordance with an embodiment of the present invention

(FIG. 23A) and a photograph (FIG. 23B) showing the distal end of a prototype of a compression-based stiffness measurement device;

FIG. 24 is a photograph of a steerable compression-based tissue stiffness measurement device in accordance with an embodiment of the present invention;

FIG. 25A is a schematic of the electrical circuit used to measure the force in the form of voltage (V);

FIG. 25B is a series of calibration curves that correlate the measured voltage (V) and force (F) using different resistance values (R);

FIG. 26 is an experimental setup showing a test of the compression-based tissue stiffness measurement device for measuring the stiffness of isolated porcine lung in accordance with an embodiment of the present invention;

FIG. 27A is a schematic and graphs showing measurement of voltage and electrical current via a force sensor and contact electrodes, respectively, to determine the tissue stiffness;

FIG. 27B is a table illustrating calculation of stiffness measurement results of porcine lung tested using a compression-based device in accordance with an embodiment of the present invention via measured electrical signals;

FIGS. 28A-28D are an overview of the robotic tissue palpation device, showing a schematic showing the application of the device in measuring the stiffness

of soft biological tissues (28A), a photograph of the robotic palpation device with an inset image showing the magnified front view of the measurement probe (28B), a schematic showing the components of the probe: contact electrodes confirm probe-tissue contact and maximum deformation, force sensor measures the force applied on the tissue by the sensor, and imaging probe provides visual information during probe navigation and measurements (28C) and a schematic showing i) undeformed tissue and ii) and fully deformed tissue (28D, wherein L_{C}: deformation length) under applied force (F_{C}), wherein maximum deformation is confirmed when the flow of electrical current (I) is generated between contact electrodes across the tissue;

FIGS. 29A-29C are photographs showing multi-directional movements of the robotic tissue palpation device, showing multiplicity photographs of catheter tip deflection movement (29A), titling movement (29B), and translational movement (29C);

FIGS. 30A-30E show a custom-built micro-optical imaging module, showing a photograph (30A) and a light-path schematic (30B) of the imaging module (C: camera, LED: light-emitting diode, TL: tube lens, F: filter, OL: objective lens, IP-A: imaging probe adapter, IP: imaging probe), a photograph showing the distal end of the imaging probe is introduced in the imaging channel of the device catheter with the inset photo showing the light is emitting from the distal end of the imaging fiber (30C), 1951 USAF and NBS 1952 test targets imaged using the imaging module (30D), and a photo of the ex vivo rat lung when the distance between the device probe and the lungs is 5 cm (30E);

FIG. 31A-31F show a schematic image (31A) and plots (31A-31F) for stiffness evaluation of gelatin-based tissue phantoms. FIG. 31A is a schematic showing the deformation of tissue sample (L_{c}: deformation length) under applied force (F_{c}) and the equation of elastic modulus. Maximum deformation is confirmed when a current (I) establishes between two contact electrodes. r: radius of sensor head, v: Poisson ratio, V_{F}: force sensor voltage, V_{E}: voltage between electrodes. FIG. 31B is a photograph and a plot showing the i) no tissue-electrodes contact and ii) full electrodes-tissue contact (i.e., maximum deformation) that is determined through a change of 3.2 volts. FIG. 31C is a graph presenting sensor's voltage versus force plots for different resistors integrated in the electrical circuit. FIG. 31D is a graph showing the measured elastic modulus (E) of tissue phantom blocks (concentration: 10%) with different thicknesses. FIG. 31E is a graph illustrating dynamic force and deformation measured from tissue phantoms with different gelatin concentrations, and FIG. 31F is a graph showing elastic moduli of tissue phantoms with different gelatin concentrations;

FIGS. 32A-32C show images (32A) and plots (32B-32C), illustrating stiffness measurements of tissue phantoms with spatially heterogenous stiffness, wherein FIG. 32A is a group of photographs showing bright-field and fluorescent images of ICG-labelled cylindrical-shaped PDMS (stiff component) at the center of a gelatin block (soft component, concentration: 10%); FIG. 32B is a photograph showing grids with 5 mm spatial resolution for scanning the tissue phantoms; and FIG. 32C shows a generated 2D stiffness map obtained following the measurement, in which E represents elastic modulus;

FIGS. 33A-33D illustrate stiffness measurements of ex vivo swine lungs, showing a photograph of the measurement setup (33A), a graph showing elastic moduli of porcine lungs with different peak inspiratory pressures (33B; PIP), bright-field and fluorescent images of the lung lobe with nodule mimic (33C), and a stiffness map of the lung cancer model (33D; E: elastic modulus; dotted circle: nodule mimic);

FIGS. 34A-34B show elastic moduli of rat (34A) and swine (34B) organs measured using a robotic palpation device (E: elastic modulus. PIP: Peak inspiratory pressure);

FIGS. 35A-35H show various motions of a robotic tissue palpation device in accordance with an embodiment of the present invention;

FIGS. 36A-36B show examples of deflection movement of a wire-driven catheter;

FIGS. 37A-37B illustrate imaging of rat organs using an optical-fiber imaging system, showing images of the ex vivo rat lungs (37A) and rat liver (37B) at different working distances (i.e., 5 cm, 2.5 cm, and 1 cm);

FIGS. 38A-38B show a force sensor circuit (38A) and sensor's calibration setup (38B);

FIGS. 39A-39B are schematics illustrating a contact electrodes circuit in accordance with embodiments of the present invention;

FIG. 40 is a schematic image showing dimensions of PDMS and gelatin block in tissue phantoms with spatially distributed stiffness;

FIG. 41 is a series of photographs showing the preparation of a lung cancer model with swine lung and silicone nodule mimic in accordance with an embodiment of the present invention;

FIGS. 42A-42F show schematic and photograph pairs illustrating stiffness measurement of rat and swine organs in accordance with embodiments of the present invention, showing the experiment setups and the position of stiffness measurement probe on the surface of rat lung (42A), rat liver (42B), swine liver (42C), swine heart (42D), swine abdominal skin (42E; ventral region); and swine abdominal muscle (42F; ventral region);

FIGS. 43A-C illustrate an image processing algorithm using Gaussian filtering for removal of core boundaries in the fiber bundle in accordance with embodiments of the present invention, showing a schematic of imaging processing steps (43A); image processing of an “S” character (43B); and image processing of lung alveoli structure (43C), wherein lung tissue sections were fixed, dehydrated, paraffinized, sliced (thickness: 5 μm), deparaffinized, and stained with Hematoxylin and Eosin stain (H&E) before imaging;

FIG. 44A is a schematic showing a dual mode probe for mechanical and electrical sensing, depicting an overview of the probe design and major components;

FIG. 44B is a schematic showing a dual mode probe for mechanical and electrical sensing, depicting an overview deformation of the tissue by the probe;

FIG. 44C is a schematic showing a dual mode probe for mechanical and electrical sensing, depicting an overview and mechanical measurements;

FIG. 45A is a schematic depicting integration of mechanical, electrical, elastography and imaging modalities in a robotic finger probe, illustrating how a probe (i.e., robotic finger) comes into contact with targeted tissue;

FIG. 45B is a schematic depicting integration of mechanical, electrical, elastography and imaging modalities in a robotic finger probe, illustrating robotic finger components;

FIG. 46 is a schematic illustration of a deployable multi-sensor probe integrated laparoscopic and robotic tools; and

FIG. 47 is a schematic diagram of an AI-enabled multi-sensor data fusion system for pathological tissue detection.

### DETAILED DESCRIPTION OF THE EXEMPLARY EMBODIMENTS

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign thereto.

Further, it should be noted that, as recited herein, the singular forms “a”, “an”, “the”, and “one” include the plural referents unless otherwise stated. Additionally, the terms “comprises” and “comprising” when used herein specify that certain features are present in that embodiment, however, this phrase should not be interpreted to preclude the presence or addition of additional steps, operations, features, components, and/or groups thereof.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

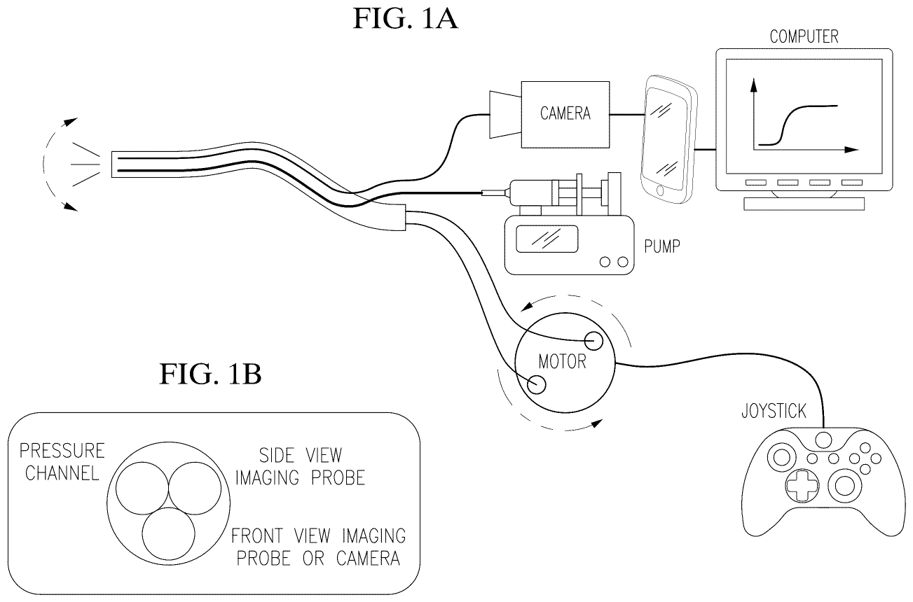

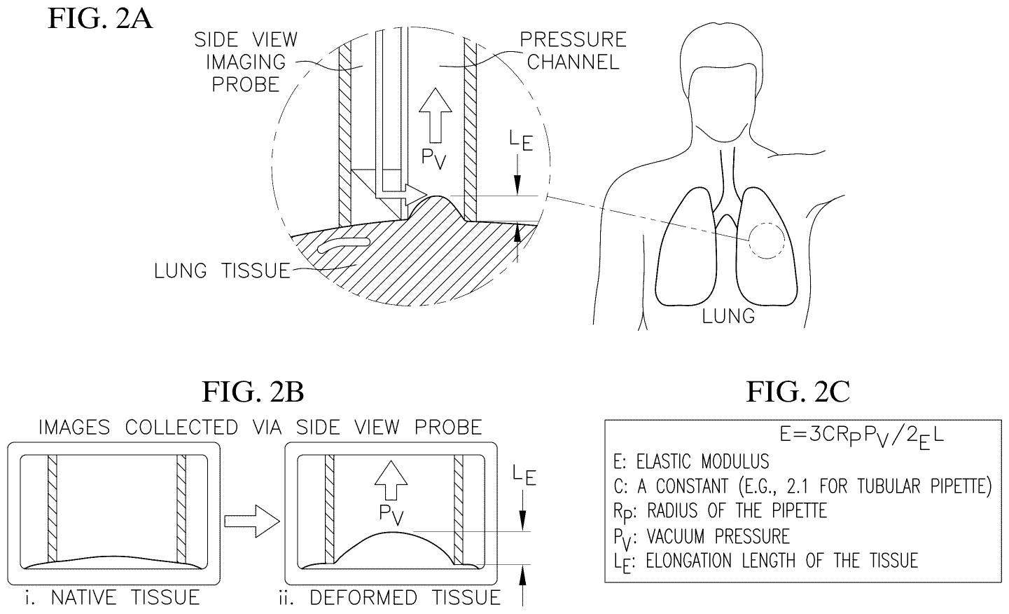

The present invention relates to a locally deployable device that is capable of non-destructive and rapid measurement of tissue and organ stiffness (see FIG. 1). In one embodiment, it is a pressure-assisted steerable device that can measure stiffness (i.e., elasticity) of local tissue within the patient's body in a localized and minimally invasive manner. A pressure channel, imaging probe(s), and camera are integrated into a steerable compartment of the device. The device is comprised of: i) a motorized steerable compartment that can be inserted minimally invasively into a patient's body; ii) a pressure network that can apply negative or positive pressure to the tissue being evaluated; iii) optical fiber probe(s) and a miniaturized camera that can guide navigation of the device and monitor the tissue deformation; and iv) a computer-based controller that regulates the pressure, analyzes collected tissue deformation data, and calculates tissue stiffness. The pressure line, imaging probe(s), and camera are integrated into the steerable compartment of the device where the tissue stiffness measurement is achieved by contacting the tissue surface of interest with the device tip. Specifically, negative pressure (P_{V}) created by a vacuum pump is applied directly to the tissue surface via the device tip while the elongation length of the tissue (L_{E}) can be determined via the side view imaging probe (see FIGS. 2A-2B).

A device made in accordance with embodiments of the present invention can be inserted the thoracic cavity via a small incision (diameter: <1 cm) created in the chest, and the device tip is placed directly onto the lung pleura surface. Negative pressure (P_{V}) is then applied via pressure channel while deformation of the lung tissue is continuously monitored via side view imaging probe. Elongation length (L_{E}) of the deformed tissue is determined from the video acquired in real time. The elastic modulus (E) of the biomaterial, which is a quantity that measures the tissue stiffness, is then determined by the formula E=3C*R_{P}*P_{V}/2πL_{E}, where C is a constant specific to the geometry of the pipette used to apply vacuum pressure to the samples, R_{P} is the radius of the pipette used for measurement, P_{V} is the vacuum pressure, and L_{E} is the elongation length of the samples. For a tubular pipette, a typical value for C is ˜2.1 (see FIG. 2C).

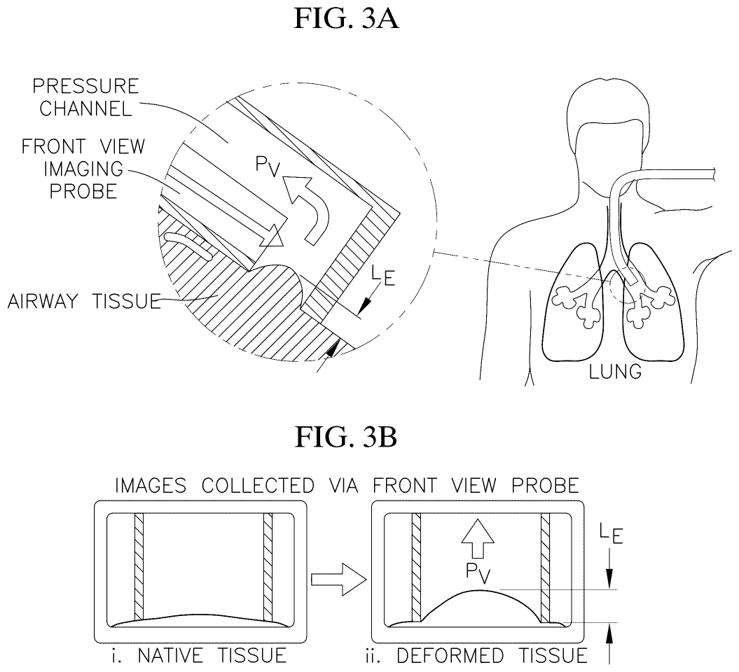

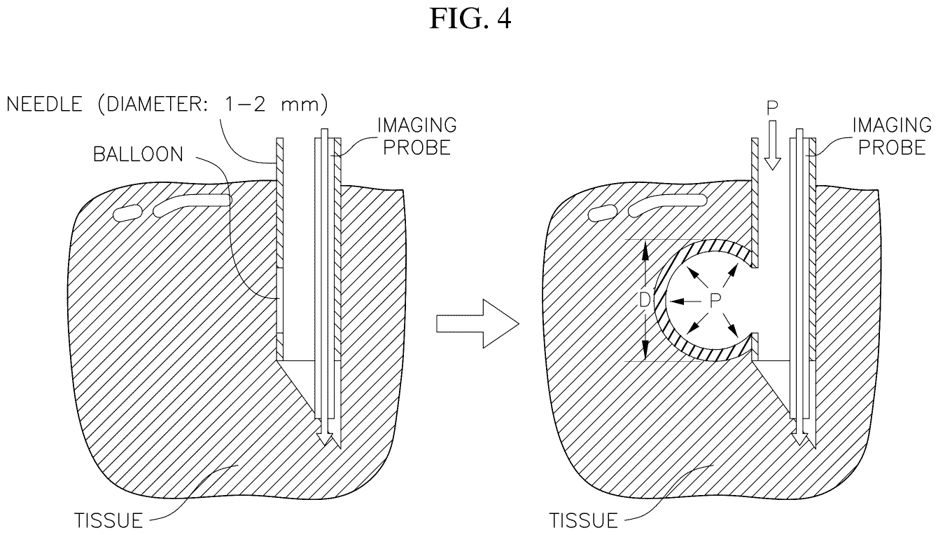

To measure luminal tissue, such as the tissues within the respiratory, gastrointestinal, or urinary tracts, the device is designed and created in a way that vacuum pressure is provided to the local tissue from the circumferential surface of the device tip (see FIG. 3A). Deformation and elongation of the tissue can be monitored via a front view imaging probe integrated into the device (see FIG. 3B). The device can be inserted into the respiratory tract via patient's mouth or nose and the device tip is positioned onto measurement site. Negative pressure is then applied while deformation of the tissue is continuously monitored via front view imaging probe. Elongation length (L_{E}) of the deformed tissue is determined from the video acquired in real time. Elastic modulus, which is a quantity that measures the tissue stiffness is calculated using an equation shown in FIG. 2C.

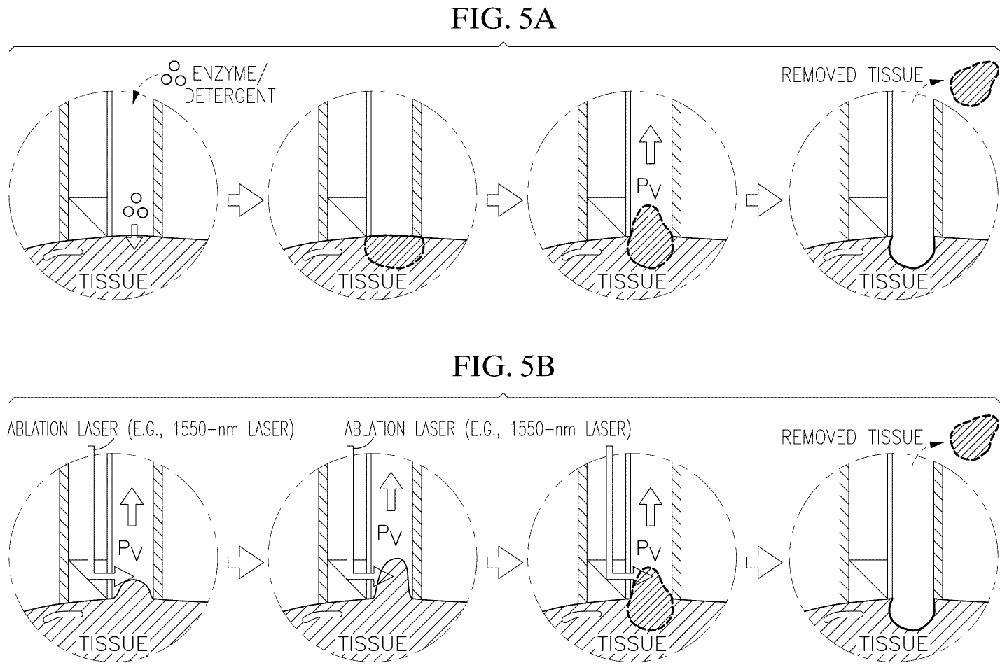

In addition, to measure stiffness of the internal tissue or biomaterial, a syringe needle (e.g., diameter: 1-2 mm) integrated with a balloon can be inserted locally (see FIG. 4). Specifically, in one embodiment, a syringe needle integrated with a balloon can be expanded via pressurized air or fluid is inserted into tissue to measure internal tissue stiffness. In this approach, the tissue stiffness (E) is obtained by relating the pressure of air or fluid (P) and the diameter of the balloon (D). P is measured using an air or liquid pressure sensor externally. D is estimated from the volume (V) of air or fluid introduced into the syringe where V=1/6πD³. While the balloon-needle is positioned internally, air or fluid (e.g., water) is provided to expand the balloon. Pressure (P) and volume (V) of the air or fluid are measured using sensors externally. Elastic modulus (E) of the tissue or biomaterial is calculated using P and V. Furthermore, the device can be used to locally deliver therapeutics to a pathologic region; take a tissue sample for in vitro analysis; and remove diseased or injured tissue. For example, fluorescent molecules (e.g., antibodies, small molecules, etc.) can be introduced via the pressure channel in order to visualize particular tissue features, such as tumors. Local tissue can also be disrupted by introducing biochemical agents (e.g., proteolytic enzymes or detergent molecules), whereby the disrupted tissue can be removed through application of vacuum pressure to the pressure channel (see FIG. 5A). In some embodiments, proteolytic enzymes (e.g., trypsin, collagenase) or detergent (e.g., SDS, CHAPS) is locally introduced to disrupt tissue where the disrupted tissue is removed by applying negative pressure (P_{V}). In other embodiments, local tissue can be removed by an illuminating laser (e.g., a 1550-nm laser) that can thermally ablate the tissue while the tissue is being stretched via the applied vacuum pressure (FIG. 5B).

### EXAMPLE 1

### Vacuum-Enabled Stiffness Measurements of Rat Lung Tissue and Soft Hydrogels

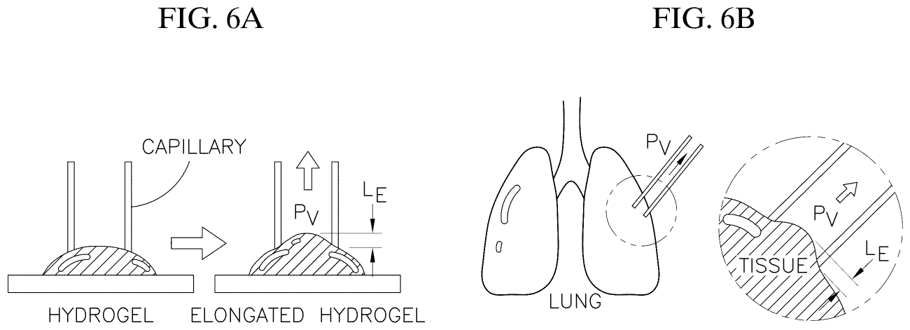

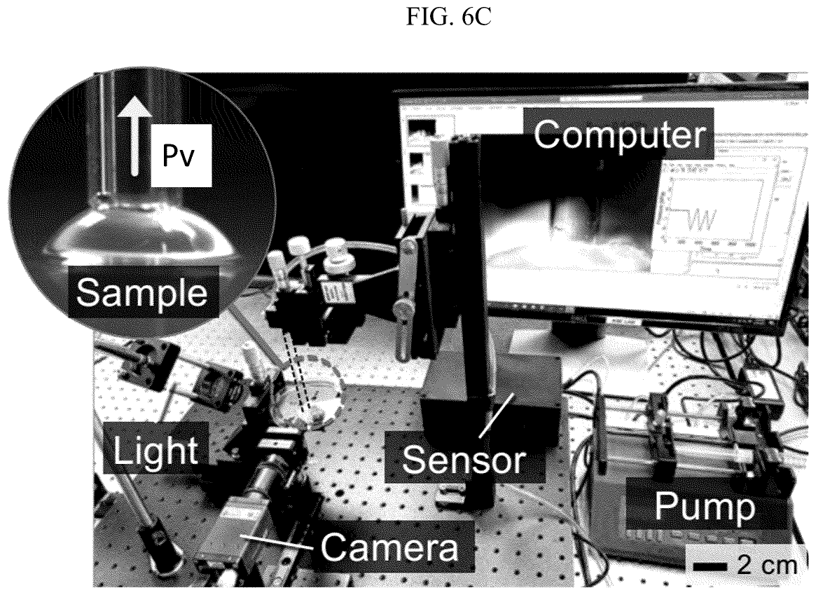

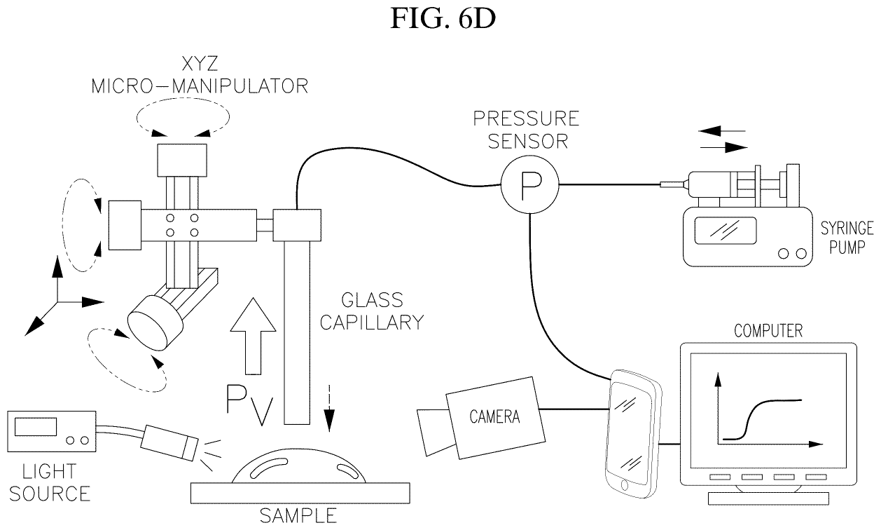

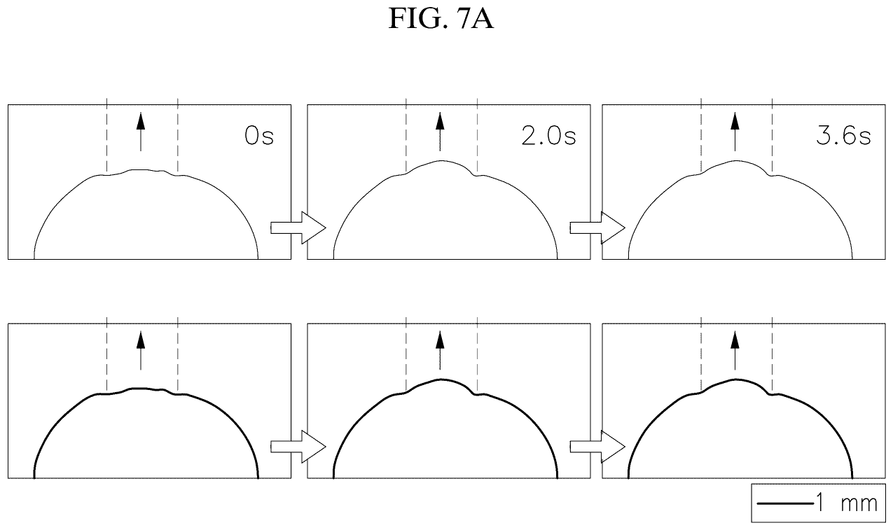

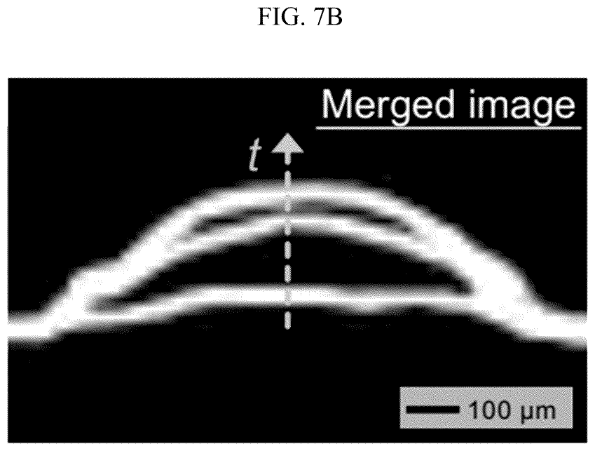

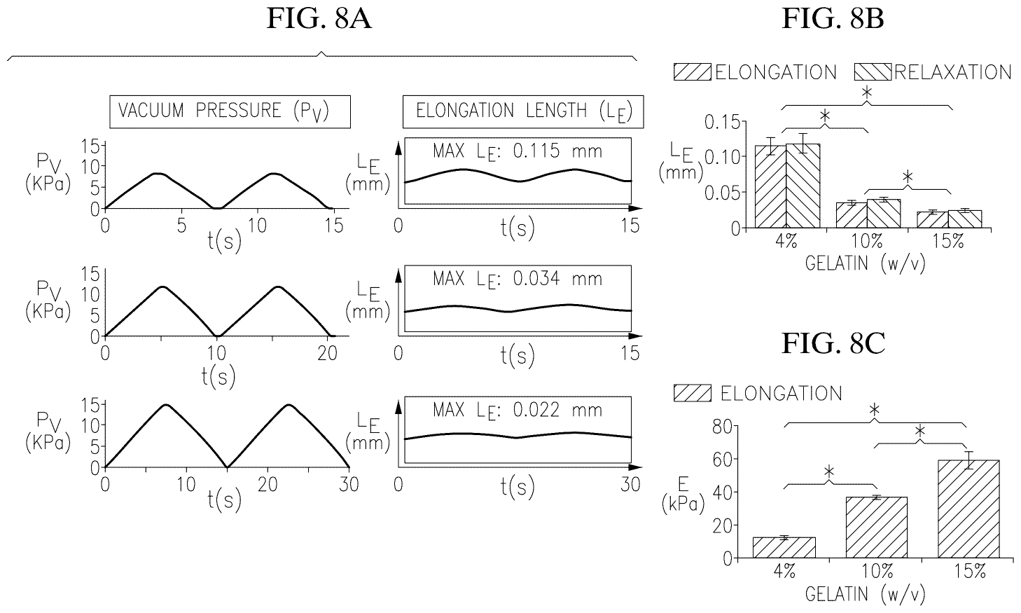

A vacuum-based platform for measurements of elastic moduli of lung tissues and soft biomaterials, such as gelatin hydrogels (FIGS. 6A-D) was developed. Functionality of this platform was tested by using a dome-shaped 4% gelatin hydrogel formed on a substrate (FIG. 7). Stiffness measurement was demonstrated using a dome-shaped 4% w/v gelatin hydrogel formed on a PDMS substrate. To enhance visibility of gel deformation, fluorescein dye was added to the gelatin. Via edge detection, the deformed shape of the gelatin was determined. Negative pressure was applied to the hydrogel via a glass capillary (inner diameter: 0.58 mm) that was gently placed on top of the gel. When the gel was exposed to −8.2 kPa, it elongated 0.115 mm. To accurately determine the deformed shape of the hydrogel, the boundary of the gel was determined via image processing, and in particular, a Canny Edge detector (FIGS. 7A-B). FIG. 7B shows a merged image of the deformed region of the gelatin showing elongation of the gel due to vacuum pressure over time. Also, gelatin hydrogels with different concentrations (i.e., 4%, 10%, and 15%) were prepared to investigate whether the platform can determine the stiffness of the hydrogels with different mechanical properties (FIGS. 8A-C). When cyclic negative pressure was applied to the gels, different elongation lengths were observed in the gels (FIG. 8A). For all samples, the rate of negative pressure increased or decreased (i.e., slope of aspiration curve) was approximately 2.25 kPa/s.

Based on the experimental parameters, such as negative pressure and tube diameter, the elongation lengths measured from the hydrogels allowed for the determination of the elastic modulus of different gels, wherein the calculated values agreed well with the values reported in the literature. For 4%, 10%, and 15% gelatins, E values were 11.913±1.008, 36.568±1.297,and 59.108±3.932 kPa, respectively (FIGS. 8B-C). Elongation length (L_{E}) of the gelatin under P_{V} of −5 kPa was determined during elongation and relaxation period of the gel, respectively. Elastic modulus (E) of each gelatin hydrogel was determined based on the measurement condition. All values represent mean ± standard deviation. *p<0.001

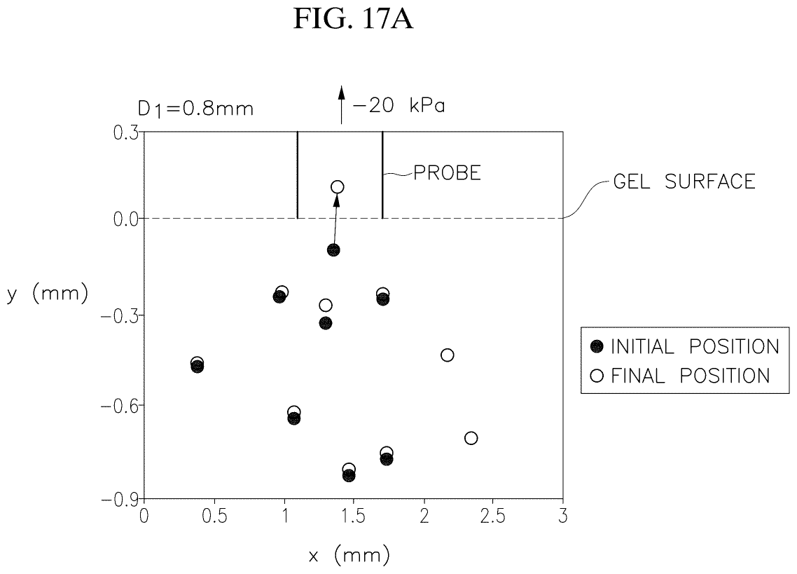

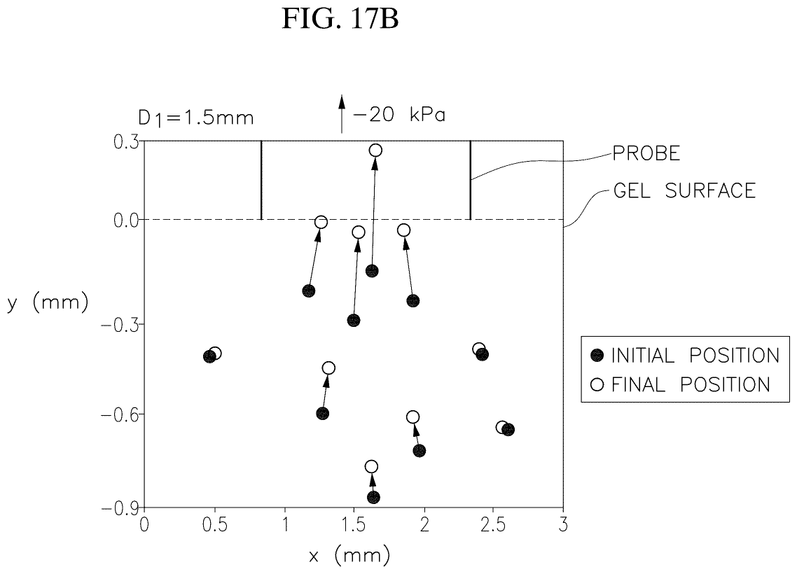

Comparisons of moduli between different gelatin groups (e.g., 4% vs 10%, 4% vs 10%, etc.) indicated significant correlation between E and gelatin concentration (p<0.001). Notably, inner diameter of the capillary probe (D_{I}) was proportional to the depth of the negative pressure energy propagated within the gel (FIGS. 17A and 17B). In particular, a larger probe (D_{I}=1.5 mm) can elongate and therefore, measure stiffness of deeper regions of hydrogel compared to a thinner probe (D_{I}=0.58 mm) which provides more superficial measurements.

Specifically, the probes were used to investigate correlation between the tube diameter and the depth of propagation of the vacuum pressure energy within a gelatin hydrogel. To visualize elongation of the hydrogel, 10-μm fluorescent particles were mixed in a 4% gelatin hydrogel and their movements were monitored using a camera. under a constant vacuum pressure of −20 kpa, the larger probe (D_{I}=1.5 mm) was able to cause movement of particles located deeper within the gel block, suggesting larger probe can allow measurement of more centrally located tissues.

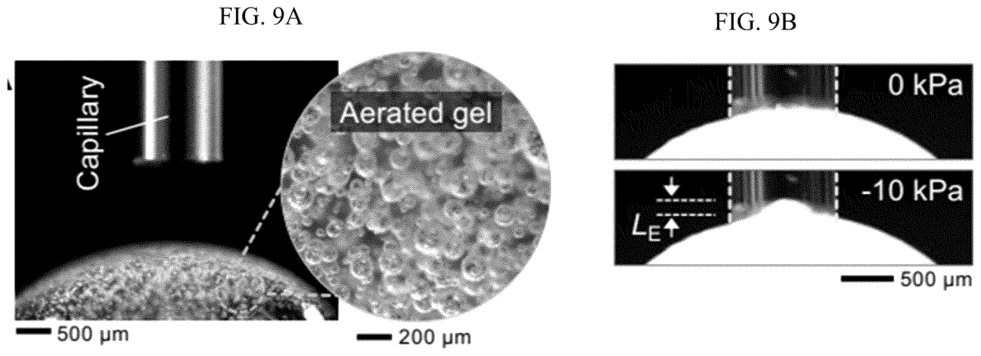

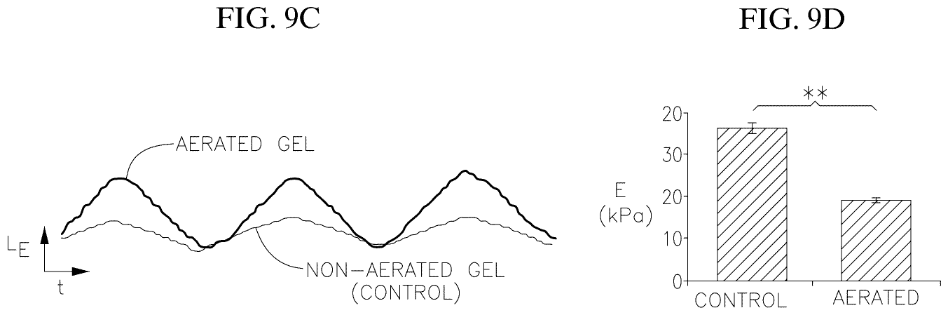

This platform was used to measure the stiffness of porous gelatin hydrogels that mimic aerated lung tissue (FIG. 9). It was then determined that the stiffness of porous 10% gelatin (E=19.065±1.298 kPa) was substantially lower than solid 10% gelatin (E=36.568±1.297 kPa) (FIGS. 9A-D). Aerated 10% w/v gelatin (FIG. 9A) was tested to investigate the correlation between gel porosity and stiffness. FIG. 9B shows images of the aerated gel showing deformed shape under vacuum pressure (P_{V}) of −10 kPa. Deformation of both aerated and nonaerated (control) 10% w/v gelatin under cyclic vacuum pressure loading over time is shown in FIG. 9C. FIG. 9D shows elastic modulus (E) of the aerated and non-aerated 10% w/v gelatin. All values represent mean±standard deviation. *p<0.001.

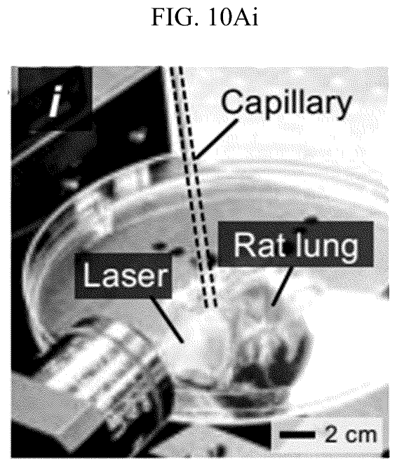

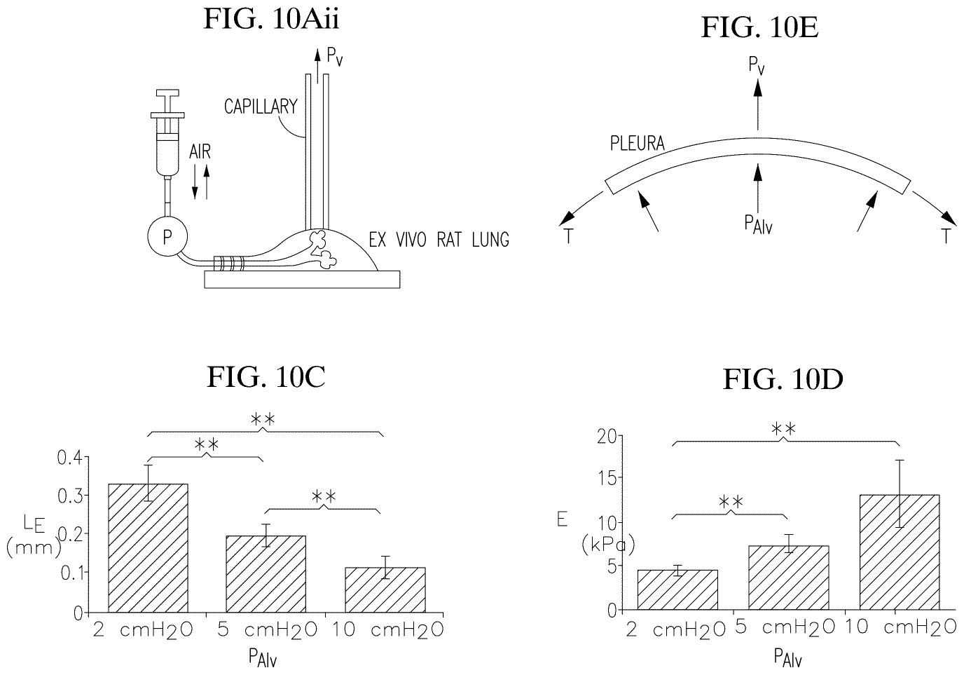

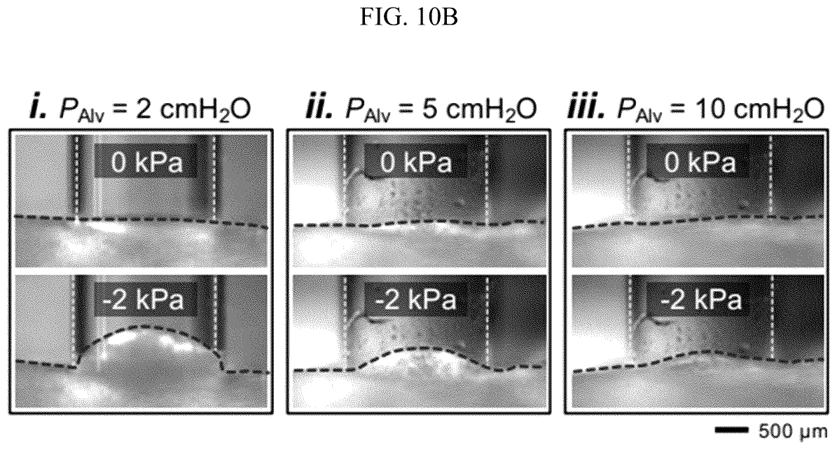

Elastic behaviors of rat lung tissues were then investigated (FIGS. 10Ai-10E). To improve the visibility of the tissue, the lung tissue was labeled with fluoresceine fluorophore prior to measurements (FIG. 10Ai). The lungs were subjected to different alveolar pressures and the stiffness of lung tissue was measured (FIG. 2A, ii; P: air pressure sensor). Notably, elongation length (L_{E}) of the lung tissue was dependent on the pressure inside the lung (P_{Alv}). As a result, for P_{Alv} values of 2, 5, and 10 cmH₂O, E values were determined to be 4.443±0.613, 7.420±1.056, and 13.174±3.854 kPa, respectively (FIGS. 10B-E). In FIG. 10B, lung tissue was subjected to vacuum pressure of 2 kPa via a glass capillary tube (inner diameter=1.5 mm) while the intra-alveolar pressure (P_{Alv}) was maintained at different pressure levels: (i) 2 cmH₂O (ii): 5 cmH₂O, (iii) 10 cmH₂O. FIGS. 10C and 10D illustrate, respectively, maximum elongation length (L_{E}) and elastic modulus E of lung tissue against different P_{Alv}. All values in FIGS. 10Ai-10E represent mean +/− standard deviation; **p<0.05.

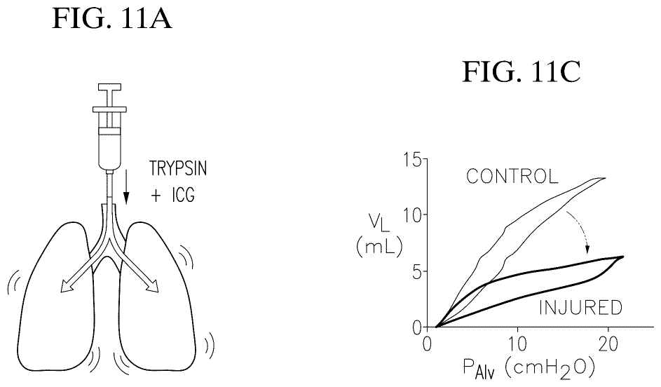

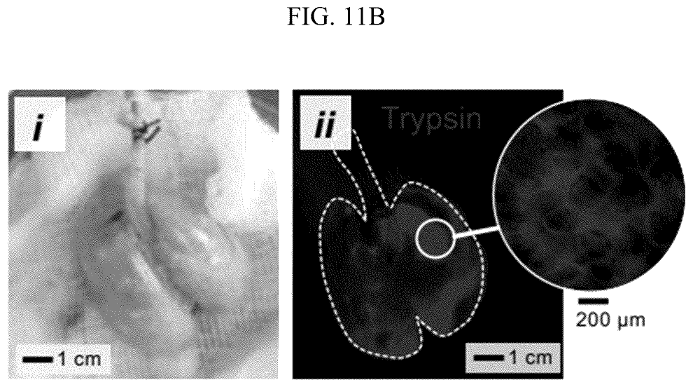

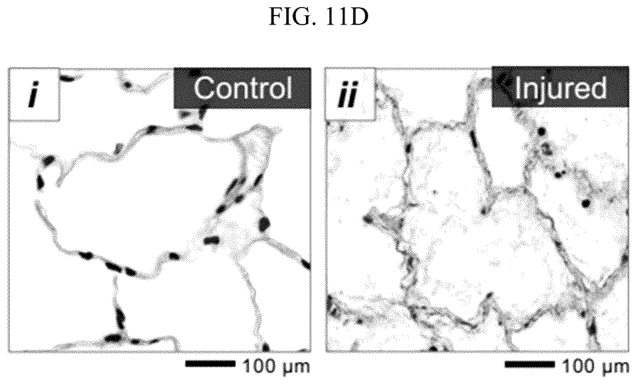

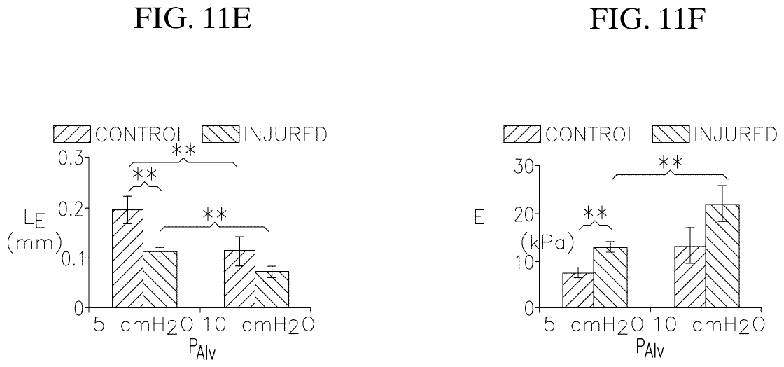

In addition, the measurement platform allowed for the quantification of the differences between the healthy and injured lung in their tissue stiffness (FIG. 11). Acute lung injury was induced by instilling 0.25% trypsin solution through the trachea of the rat lung. To visually confirm delivery of the solution in the lung, indocyanine green (ICG) fluorescent dye molecules were added to the trypsin solution (FIGS. 11A-B). FIG. 11B shows (i) explanted rat lungs before injury and (ii) distribution of trypsin/ICG (red) visualized via NIR imaging. FIG. 11C shows pressure-volume (PV) curves of the lung before and after injury, which were obtained by measuring the intra-alveolar pressure (P_{Alv}) and volume (V_{L}) of air inspired or expired through the trachea of the lung using a small animal ventilator. The compliance of the injured lung substantially decreased (FIG. 11C), while histologic analysis of the injured tissue showed severe damage in the alveolar tissues (FIG. 11D). Notably, stiffness of the injured lung was greater than healthy lung (FIGS. 11E-F). The increase in stiffness of the injured lung is likely due to dysregulation of native pulmonary surfactant in the alveoli caused by displacement and dilution by the enzyme solution. All values in FIG. 11 represent mean +/− standard deviation; **p<0.05.

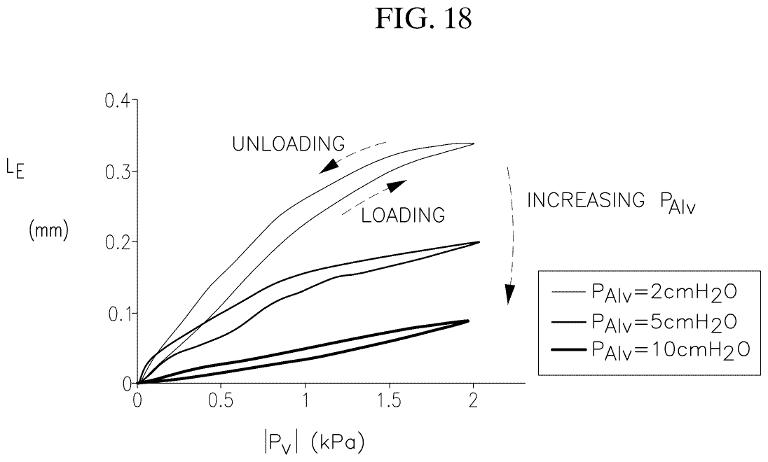

Intact rat lungs were also used to investigate whether the elastic modulus of lung tissue can be measured non-destructively using the vacuum-assisted method. During measurements, the intra-alveolar pressure of the lung (P_{Alv}) was maintained at a constant level (2, 5, 10 cmH₂O) without ventilating lungs to minimize motion-induced measurement error and prevent tissue damage that could be caused by uncontrolled contact of the capillary probe with the tissue surface. To improve visualization of tissue deformation, a 488-nm laser was used to directly illuminate the lung pleural tissue labeled with fluoresceine molecules prior to measurements. The pressure inside the lung (P_{Alv}) was controlled and monitored using a syringe connected to a pressure sensor. Photographs showed deformation of lung tissue under a negative pressure (P_{V}) of −2 kPa via a capillary tube (inner diameter: 1.5 mm) while different P_{Alv} (2, 5, 10 cm H₂O) were maintained within the lung. Further, the elongation length of the lung tissue (L_{E}) was measured, while P_{V} was increased and decreased to imitate the stress-strain (i.e., pressure-volume) measurements of lung (FIG. 18) measured L_{E} varied nonlinearly in response to P_{V} while the loading-unloading curve exhibited hysteresis which is a unique behavior of viscoelastic materials, including lung tissue.

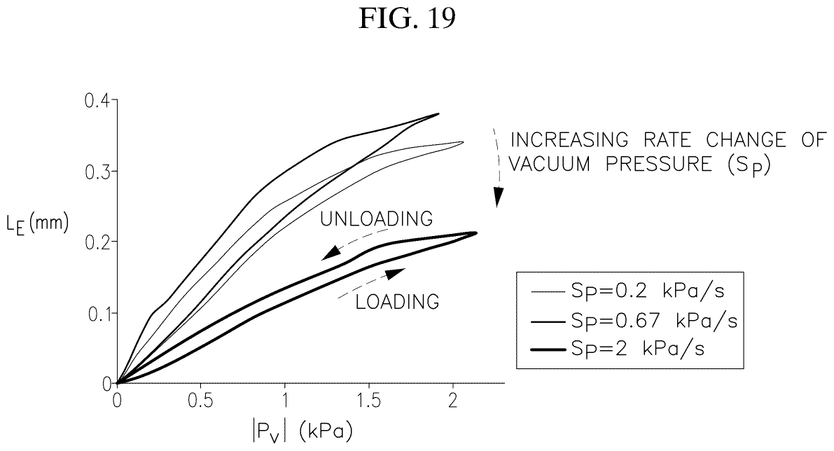

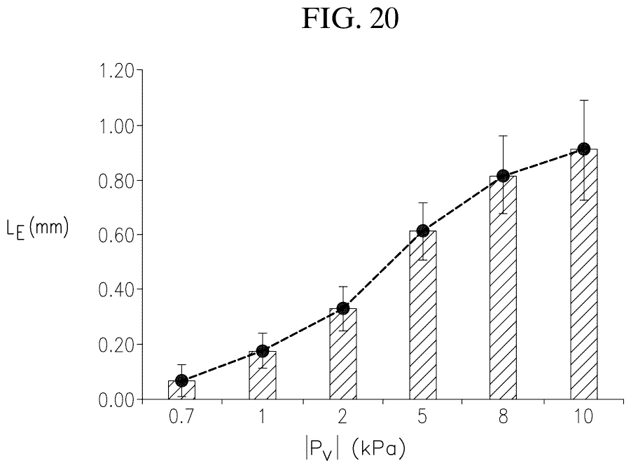

FIG. 18 also shows the effects of vacuum pressure (P_{Alv}) on tissue elongation length (L_{E}): for rat lung maintained at different P_{Alv}, L_{E} were measured continuously while P were varied between 0 and 2 kPa. L_{E} and P_{V} were nonlinearly related and the curves exhibited hysteresis. Increasing P_{Alv} resulted in reduced L_{E} due to increased forces within the alveolar network and pleural layer. In addition, L_{E} was measured while varying rate of change of vacuum pressure magnitude (|P_{V}|) (i.e., 2 kPa/s, 0.66 kPa/s, and 0.2 kPa/s) (FIG. 19). Notably, increased rate change of |P_{V}| resulted in reduced L_{E} indicating viscoelastic deformation pattern of the lung. It was further confirmed that the non-linear deformation of lung tissue was present by measuring L_{E} while increasing |P_{V}| from 0.7 to 10 kPa in a step-wise manner (FIG. 20). At lower pressure (|P_{V}|<2 kPa), L_{E} increased rapidly with |P_{V}|, while further increasing magnitude of the negative pressure (e.g., |P_{V}|>5 kPa) resulted in decreased rate of tissue elongation, highlighting non-linear correlation between L_{E} and |P_{V}|.

With reference to FIG. 19, effects of the rate change (S_{P}) of magnitude of the negative pressure (|P_{V}|) on tissue length (L_{E}) is shown. L_{E} were continuously recorded while varying (|P_{V}|) between 0 and 2 kPa. The obtained curves showed a nonlinear relationship between L_{E} and (|P_{V}|) and hysteresis was observed as L_{E} were generally greater during unloading than loading. Furthermore, changing the pressure values rapidly (i.e., increasing S_{P}) resulted in reduced overall L_{E}. Together, the results confirmed viscoelastic behavior of the lung parenchymal tissue.

With reference to FIG. 20, the correlation between vacuum pressure |P_{V}| and tissue elongation length (L_{E}) is illustrated. L_{E} were measured against different |P_{V}| between 0.7 and 10 kPa. While L_{E} and |P_{V}| were approximately linearly related below 2 kPa, L_{E} increased slowly at higher |P_{V}| (e.g., above 5 kPa) suggesting that greater negative pressure is needed to deform load-bearing lung tissue. Because of this nonlinear relationship between L_{E} and |P_{V}| |P_{V}|=2 kPa was used throughout the tests to compare stiffness of different lungs.

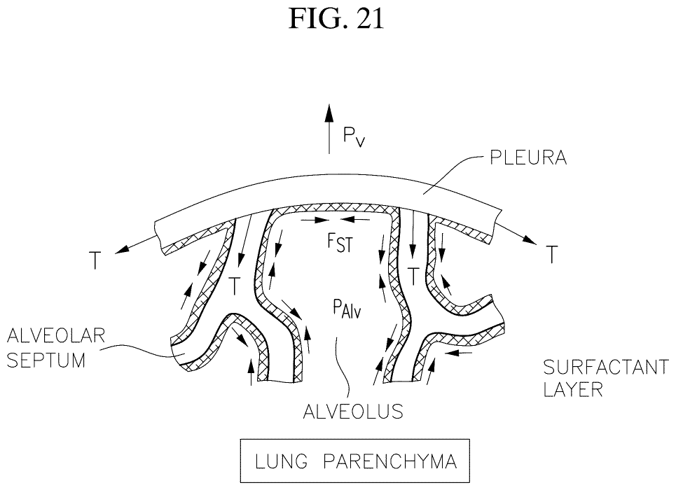

Notably, as pressure inside the lung increased, L_{E} decreased. For P_{Alv} of 2, 5, and 10 cmH₂O, L_{E} were 0.33±0.05, 0.20±0.03, and 0.12±0.03 mm and E were 4.4±0.6, 7.4±1.1,and 13.2±3.9 kPa, respectively. The results indicate that there was an interaction between P_{Alv} and E (0.2 vs 0.5 kPa: p=0.005; 0.2 vs 1.0 kPa: p=0.0058; 0.5 vs 1.0 kPa: p=0.067). Such alveolar pressure dependence is due to the changes in tension (T) within the pleural layer and alveolar septal network with respect to the air pressure inside. As the lung volume increases, the collagen fibrils in the tissue become stress-bearing while their waviness is lost, leading to increased tissue elastic modulus. Therefore, as the internal pressure of the lung elevates by increasing the volume of air in the lung, tension in the lung tissue also increases, requiring a greater vacuum pressure to stretch the lung tissue against the resisting tensile force (FIG. 21) Lung stiffness measured using the vacuum-based method were within ranges reported in the literature, obtained using a broad range of mechanical testing methods, including indentation, atomic force microscopy, and tensile testing.

### EXAMPLE 2

### Optical Fiber Imaging Probe for Monitoring Tissues

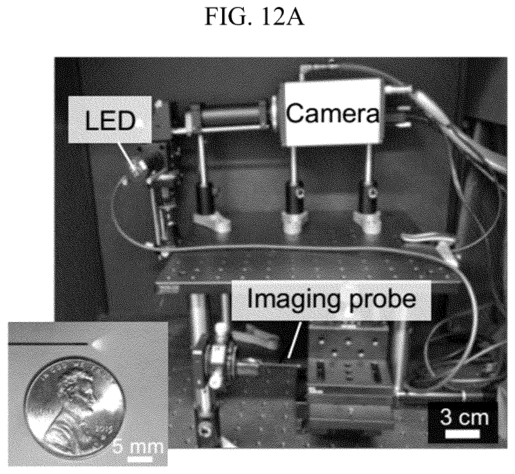

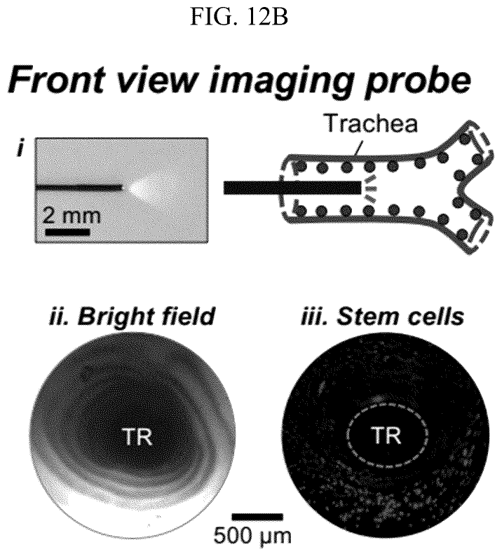

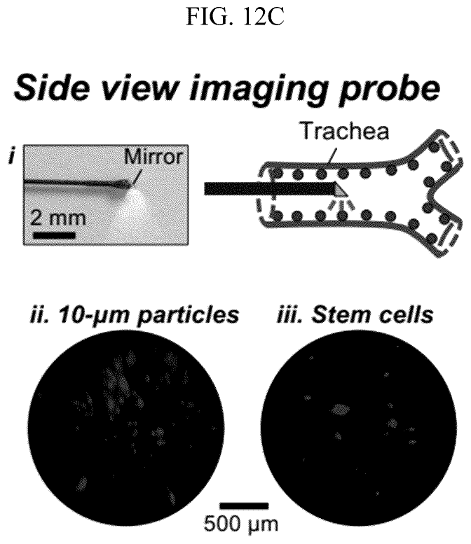

A custom-built imaging platform was created utilizing optical imaging probes (both front view and side view probes) that were capable of imaging local tissues in bright-field and fluorescence (FIG. 12). The imaging system is comprised of a scientific camera, an LED or laser light source, optical filters, and an optical imaging probe (FIG. 12A). Using this imaging system, visualization of the interior of the rat lung was demonstrated. In particular, both bright-field and fluorescent imaging of the inside of the rat trachea were achieved using a front view imaging probe (FIG. 12B). Using a side view imaging probe, the luminal surface of the rat trachea was visualized with reduced optical distortion (FIG. 12C).

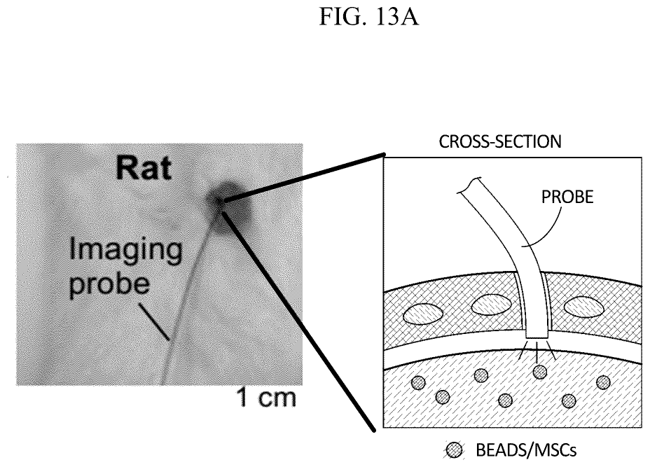

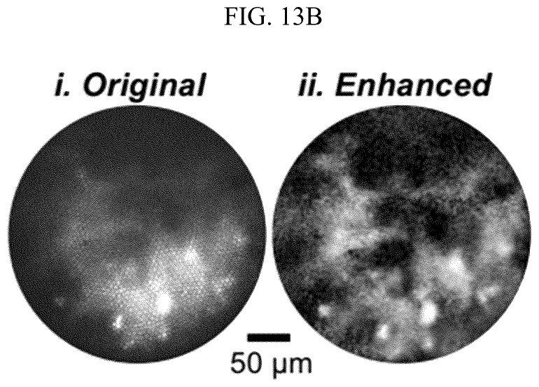

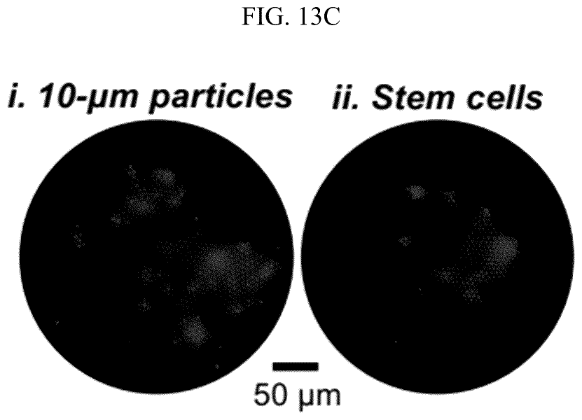

For fluorescent imaging using both imaging probes, red 10-μm microparticles or mesenchymal stem cells were implanted. In addition, this imaging system was used to visualize the rat lung in situ (FIG. 13). Imaging of the rat lung was achieved by inserting the probe into the thoracic cavity through a small incision created in the chest of the animal (FIG. 13A). Bright-field images of the rat lung were obtained through this imaging approach, wherein a front view imaging probe was placed near the lung pleura. By enhancing the contrast of the image, individual alveoli could be visualized clearly (FIG. 13B). Furthermore, this imaging system allowed fluorescent imaging of the rat lung in situ wherein the 10-μm particles and mesenchymal stem cells (red) introduced into the alveolar space of the rat lung were clearly visualized (FIG. 13C) through the thin pleural layer. The results suggest that this imaging system can be used to locally inspect tissue deformation and measure elongation of the deformed tissue via vacuum pressure.

### EXAMPLE 3

### Construction of a Steerable Catheter for Integration of Vacuum Channel and Imaging Probe

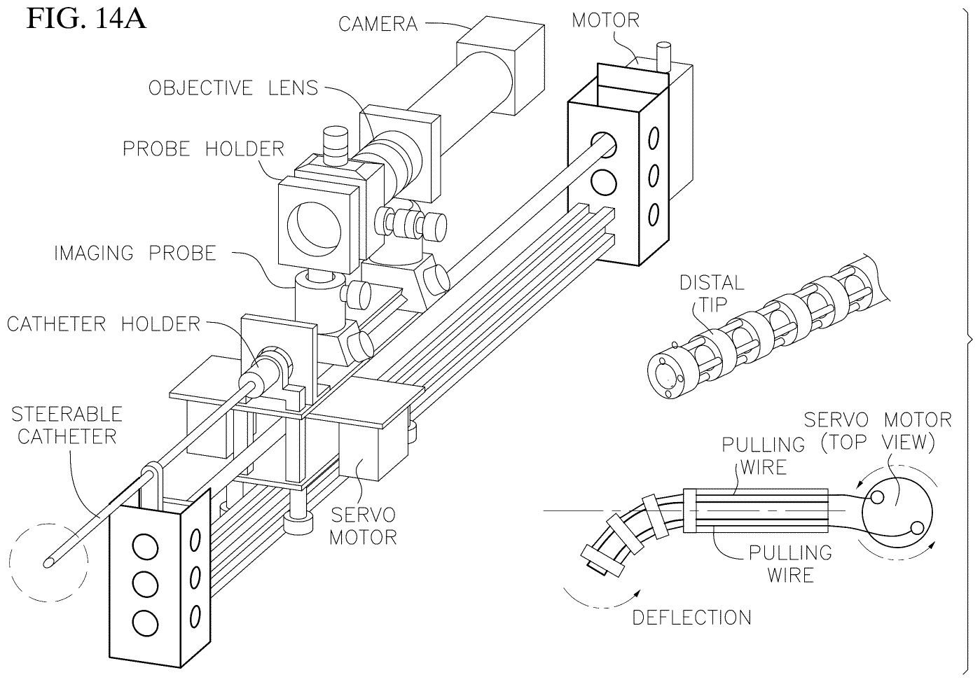

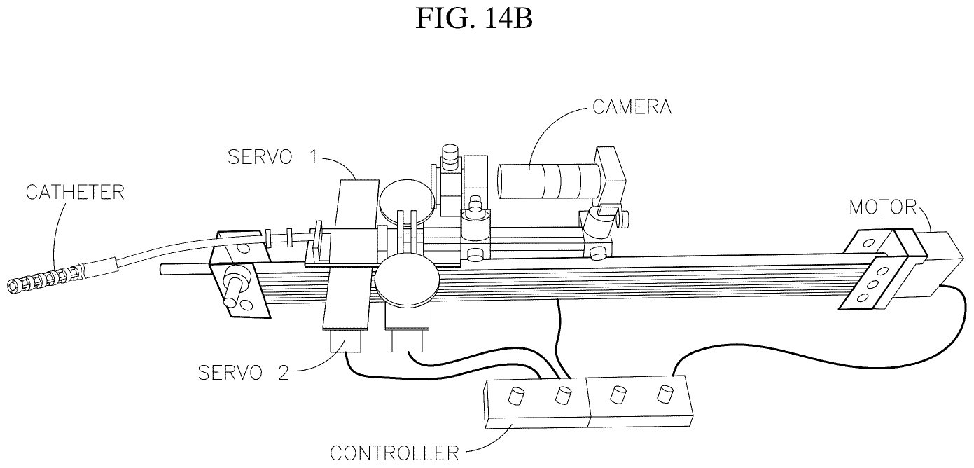

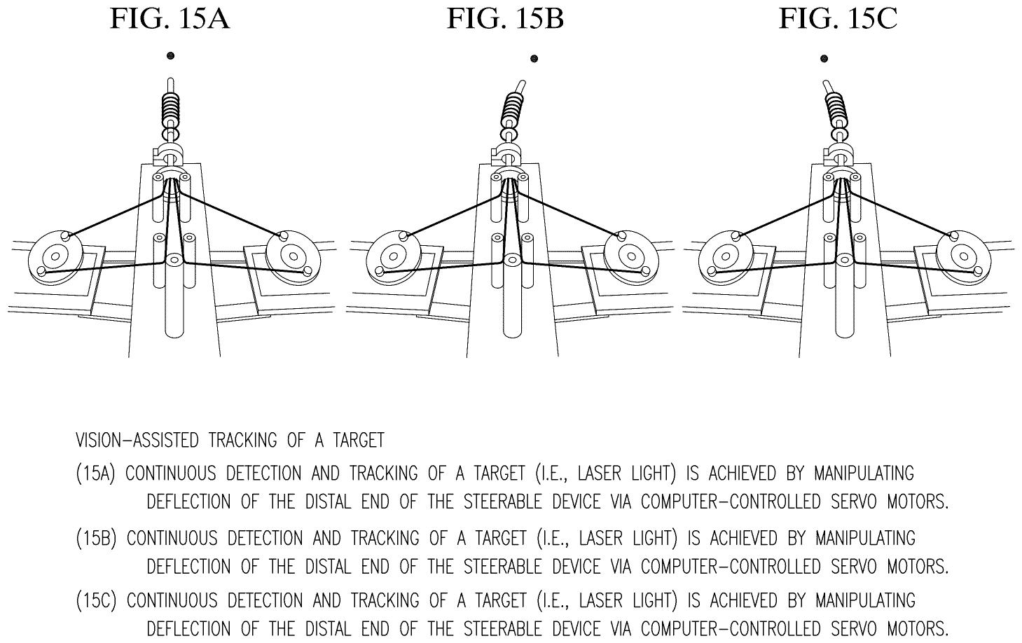

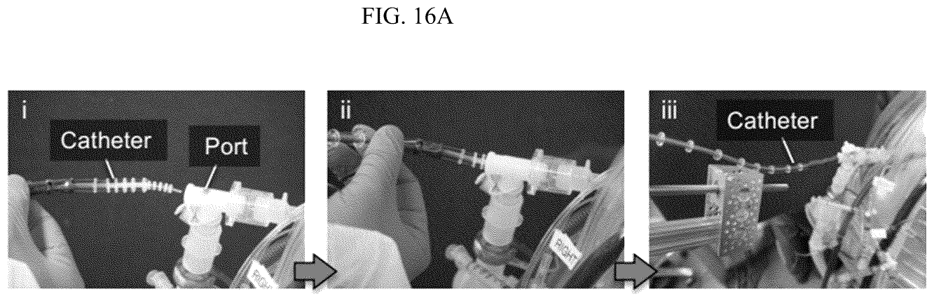

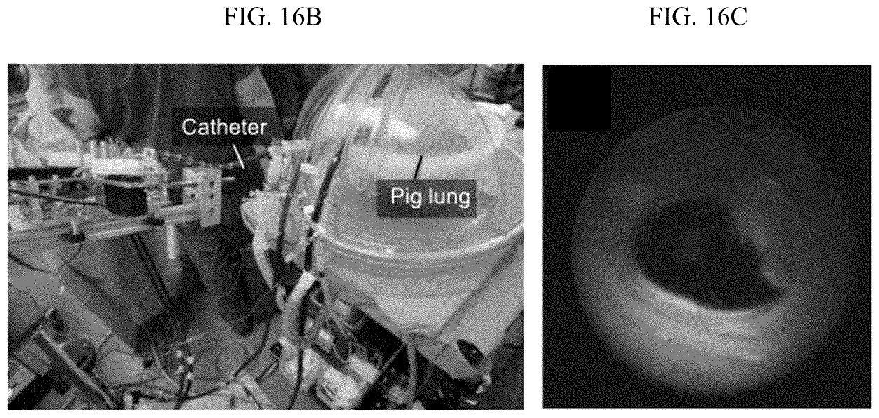

A motorized steerable catheter device was created, into which the vacuum channel and imaging probe can be integrated for localized tissue stiffness measurements (FIG. 14). As shown in the 3D rendering image of the device, servo motors, pulling wires, and a motor controller integrated together collectively control deflection and translational movement of the device tip (FIG. 14A). A protype of the device was constructed, which comprises three servo motors, a motor controller, and an optical fiber imaging probe that is capable of vision-assisted three-dimensional navigation in space (FIG. 14B) In the prototype, the steerable catheter is integrated with servo motors and a motor controller (i.e., joystick) that can control movements of the device. Using this device, vision-assisted continuous tracking of an object was demonstrated by manipulating deflection movements of the device using a motor controller via computer-controlled servo motors (FIGS. 15A-C). Furthermore, the feasibility of the device was demonstrated in visualization of the airway lumen using explanted swine lungs (FIGS. 16A-C). Via an access port, the device was inserted into the lung through the trachea (FIGS. 16A-B). The interior of the airway of the lung was visualized using the device inserted (FIG. 16C).

The invention described herein facilitates simultaneous tissue evaluation and removal. While similar technologies/devices are limited to assessment of tissue stiffness, the present invention can allow not only localized tissue evaluation, but also tissue biopsy or ablation (FIG. 5). The multi-functional approach of the present invention can reduce the number of procedures required for the patients by allowing simultaneous diagnosis and treatment during a single intervention.

Regarding computer vision-assisted accurate determination of tissue deformation, it is important to determine the exact deformed shape of the tissue caused by exposure to vacuum pressure. In the inventive device, the shape of the tissue before and after the vacuum-induced deformation can be accurately determined in real time via computer-vision enabled boundary detection (or edge detection) methods (FIGS. 7-8). The computer-assisted tissue boundary detection can allow accurate measurement of the tissue elongation from which stiffness of the tissue being evaluated can be calculated.

Regarding in situ fluorescence tissue visualization, to enhance the quality of images acquired during tissue deformation for accurate assessment of tissue deformation, the present invention can be integrated with in situ fluorescence imaging capability (FIGS. 6A-D), wherein P_{V}: vacuum pressure. L_{E}: elongation length, and P: Air pressure sensor. Due to autofluorescence, human tissues normally emit green lights when they are exposed to blue light. Accordingly, the local tissue being evaluated using the present device is illuminated with blue laser light (wavelength of approximately 488 nm), while green light generated by the tissue is collected via a camera connected to the device externally. The acquired images are processed via computer algorithms to determine the tissue boundary, elongation length, and tissue stiffness. In addition, fluorescent molecules (e.g., fluoresceine, rhodamine, tagged antibodies) that can label the tissue and/or specific tissue features fluorescently can be introduced through the device lumen to further improve the visibility of the tissue and identify particular tissue features and enhance the accuracy of tissue deformation and elongation.

### EXAMPLE 4

### Alterations in Stiffness Caused by Alveolar Disruption

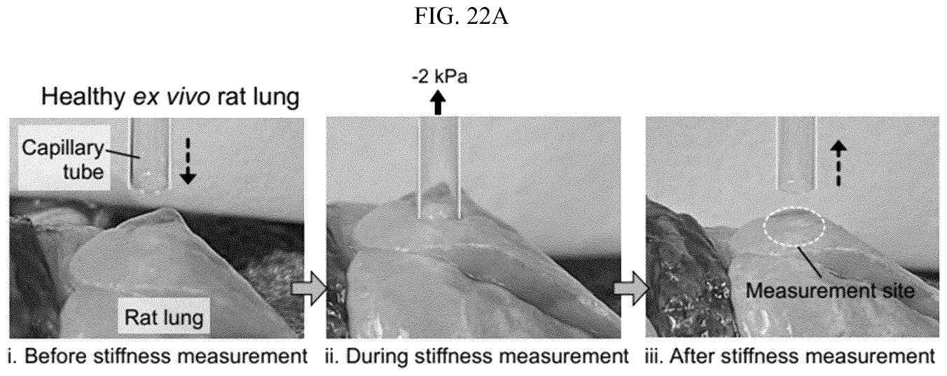

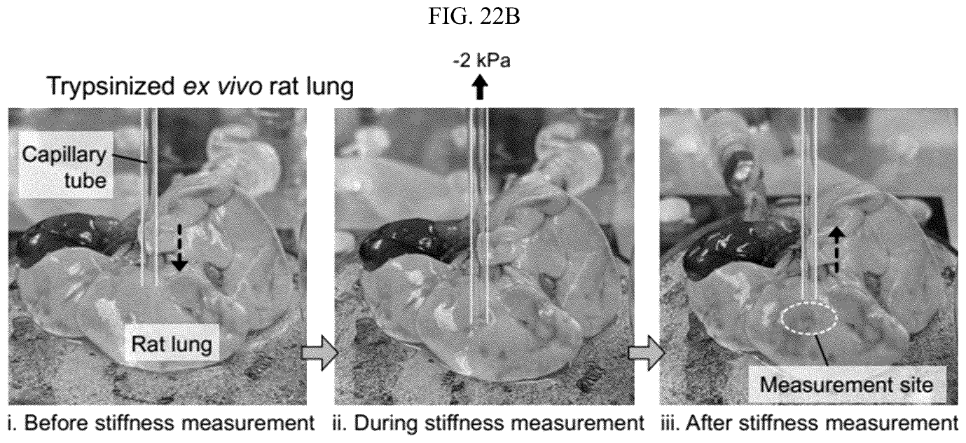

It was further investigated whether changes in the stiffness of lung parenchymal tissue caused by enzymatic disruption can be detected using the approach of the present invention. To induce acute tissue disruption, the lung was exposed to an enzymatic solution (i.e., trypsin) that can dislodge epithelial cells from ECM and further disrupt the surface tension in the alveolar space (FIG. 11). The goal of trypsinization was not to mimic stereotypical presentation of any one pathology, but rather to assess the ability of the vacuum-assisted method to quantify changes in lung tissue stiffness. The rat lung was instilled with 0.25% trypsin (1 mL) with ICG dye through the trachea and incubated for 10 min. Distribution of the trypsin solution within the respiratory tract of the lung was confirmed through visualization of trypsin/ICG via NIR imaging of the lung. Static compliance (C_{S}) of the whole lung was monitored before and after the trypsin challenge by measuring its pressure-volume relation, where air pressure (P_{Alv}) and lung volume (V) were measured using a custom-built sensor module. Static compliance (C_{S}) was calculated by C_{S}=TV/(P_{Plat}−PEEP), where TV is tidal volume, P_{Plat} is plateau pressure, and PEEP is positive end-expiratory pressure. While the healthy lungs (control) displayed high static compliance (0.70 mL/cmH₂O), damaged lungs showed substantially reduced compliance (0.29 mL/cmH₂O), as trypsinized lungs required significantly greater pressures when ventilated with the same air volume (FIG. 11C). Histological analysis via H&E staining of the trypsinized lung showed excessive accumulation of fluid and debris in alveolar spaces with substantial reduction of intact cells and decreased number of nuclei compared to control lungs (FIG. 11D). In the trypsinized lungs, maximum elongation lengths under P_{V} of −2 kPa were 0.11±0.01 and 0.07±0.01 mm, respectively, for 5 and 10 cmH₂O of P_{Alv} (FIG. 7A). As a result, elastic moduli of the enzymatically damaged lungs were determined to be 12.8'1.0 and 21.7±3.9 kPa, respectively, for 5 and 10 cmH₂O of P_{Alv}, indicating 73.1% and 64.6% increases in tissue stiffness compared to the control (healthy) lungs under the same measurement conditions. The significant increase in E following proteolytic tissue disruption, in particular for P_{Alv} of 5 cmH₂O (p=0.003), suggests that the intrinsic deformation mechanics and stiffness of the lung tissue parenchyma was compromised following trypsin challenge. Further, in the damaged lungs, the effects of P_{Alv} on E were considerable (p=0.02), highlighting the influence of measurement conditions such as intra-alveolar pressure on tissue characterization. Notably, no physical damages to the lung parenchyma or pleura (e.g., blistering or rupturing) were observed in the visceral pleural layer of the lungs following the vacuum-based measurements (FIG. 22). FIG. 22 illustrates both healthy (FIG. 22A) and trypsinized rat lungs (FIG. 22B). Both were inspected visually for physical damage, such as blistering, that could occur due to negative pressure applied to the pleural surface. Photographs of lung taken during vacuum-based stiffness measurements (P_{V}=−2 kPa) showed that the pleural layer of the lungs remained intact with sign of physical damage created by the vacuum applied.

### EXAMPLE 5

### Compression-Based Palpation Device

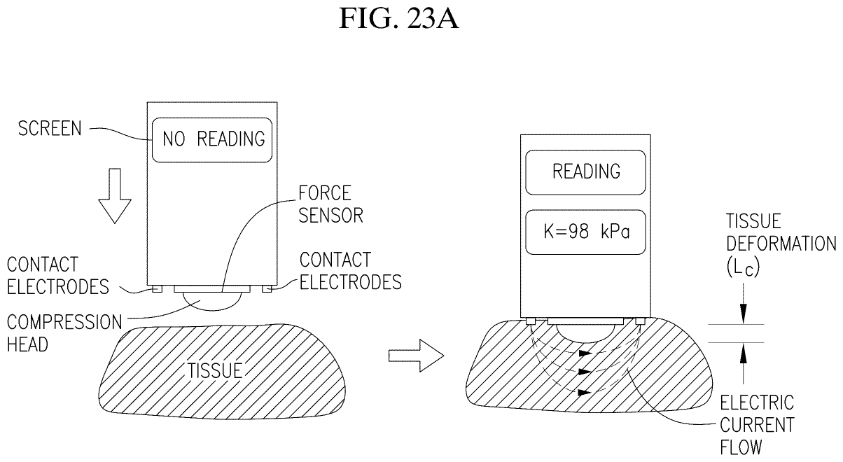

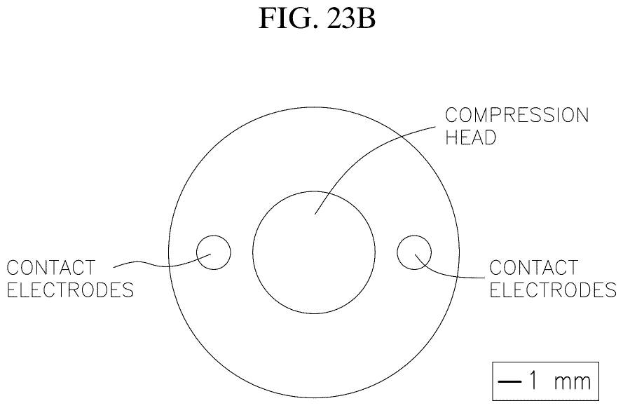

FIG. 23A is a schematic representation of compression-based tissue palpation device and its operation procedure. Contact electrodes ensure tight contact between the device tip and tissue during measurement as no electrical signal is detected if tissue compression is incomplete. A force sensor incorporated at the device tip measures the compression force (F) applied to the tissue. Compression head with a fixed and known height defines the tissue deformation length (L_{C}), allowing consistent tissue deformation across measurements. Tissue stiffness (K) is then calculated by relating F and L_{C}. FIG. 23B is a photograph showing the front-view of the distal end of a prototype for a compression-based stiffness measurement device.

In an embodiment, a device made in accordance with an embodiment of the present invention features a compression head with a fixed and known height that defines the tissue deformation length (L_{C}), allowing consistent tissue deformation across measurements (FIGS. 23A-23B). The use of contact electrodes ensures tight contact between the device tip and tissue during measurement, as no electrical signal will be detected if tissue compression is incomplete. The force sensor incorporated at the device tip measures the compression force applied to the tissue. Such a device can be created into a portable, hand-held configuration for rapid assessments of external tissues or a steerable configuration for in vivo tissue assessments.

In such embodiments of the present invention, a measurement procedure can be followed: First, during tissue stiffness measurement, the device tip is gently pushed against the tissue. Next, tissue deformation continues until an electrical signal is detected via the contact electrodes. Finally, Tissue stiffness is calculated based on the tissue deformation (L_{C}) and force (F) recorded at the time of electrical signal detection.

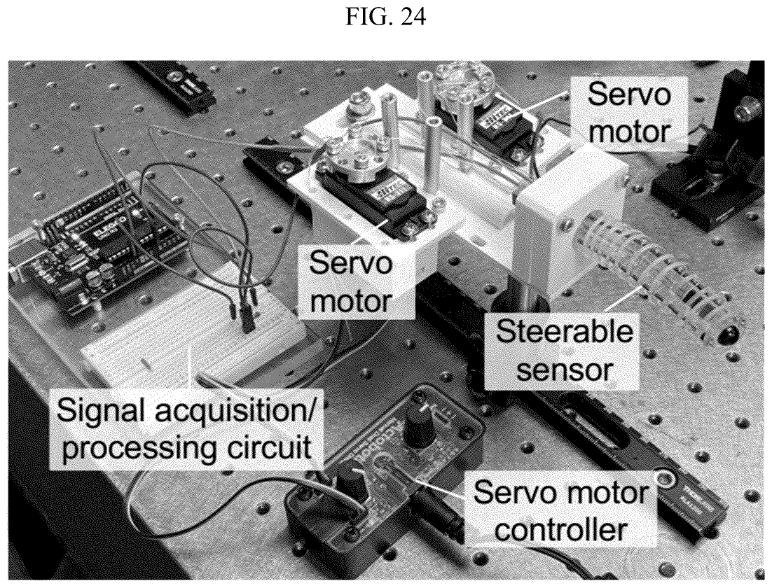

FIG. 24 is a photograph showing a prototype of a steerable compression-based tissue stiffness measurement device. The deflection and translational movement of the device is controlled by servo motors and a motor controller. Compression force measured via the force sensor and the electrical signal detected via the contact electrodes integrated at the distal end of the device is recorded and processed by an electric circuit.

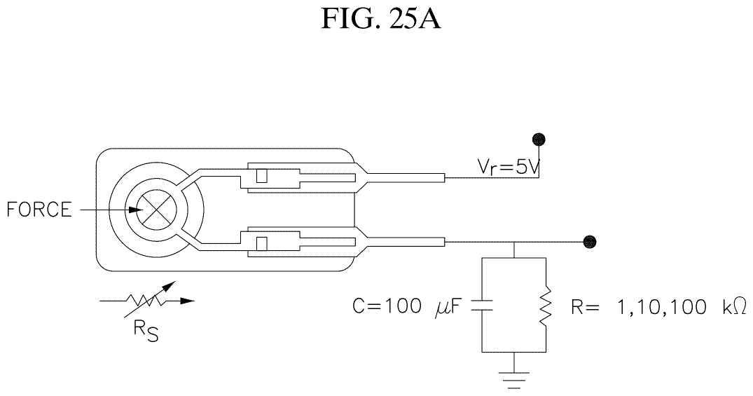

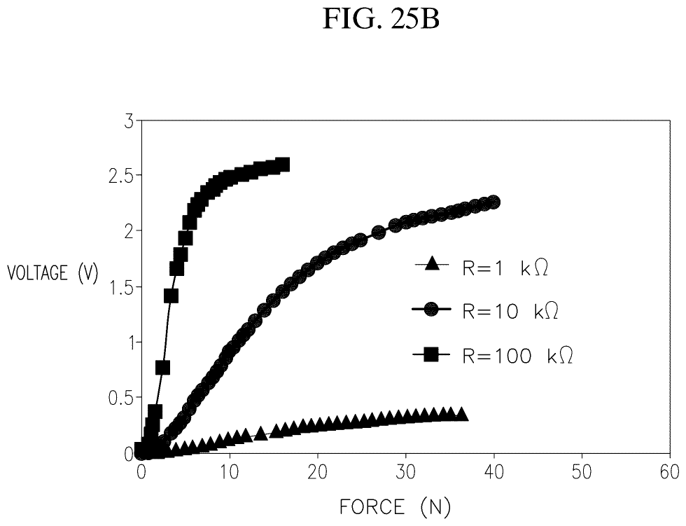

FIG. 25A is a schematic of the electrical circuit used to measure the force in the form of voltage (V). FIG. 25B is a series of calibration curves that correlate the measured voltage (V) and force (F) using different resistance values (R).

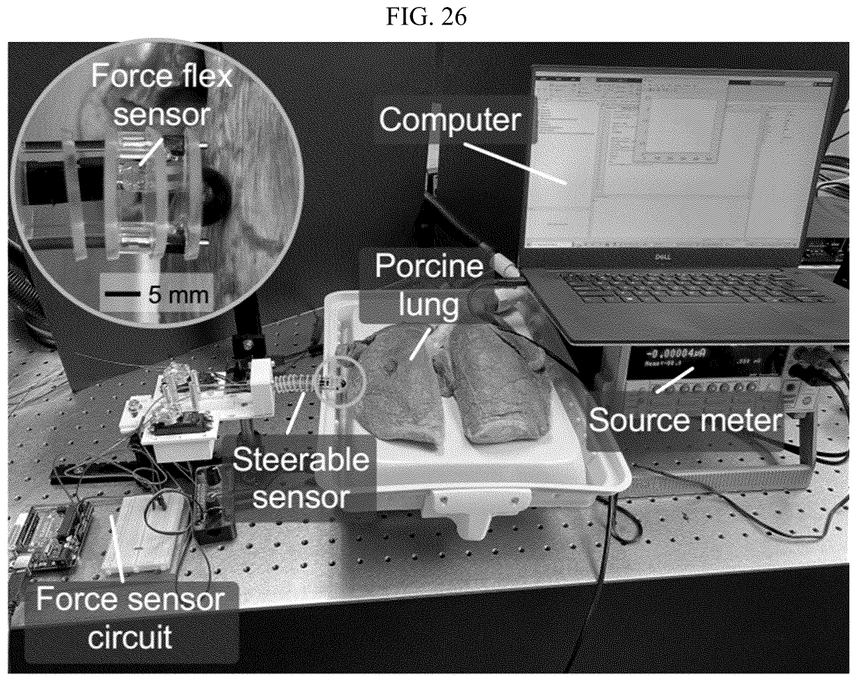

FIG. 26 is an experimental setup showing test of the compression-based tissue stiffness measurement device for measuring the stiffness of isolated porcine lung.

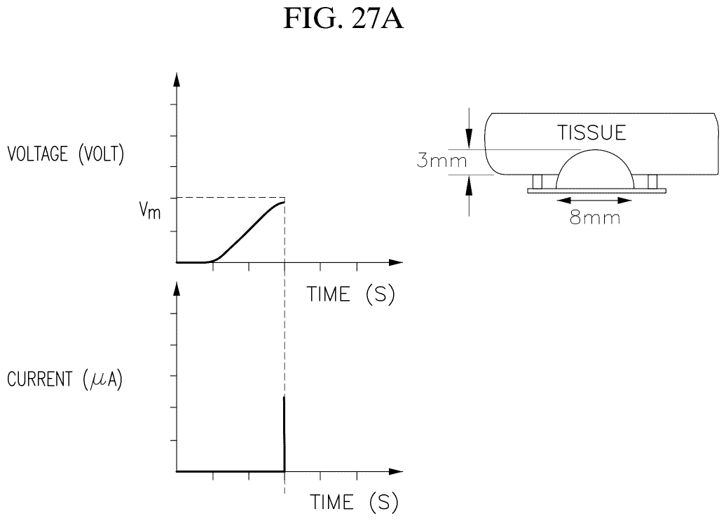

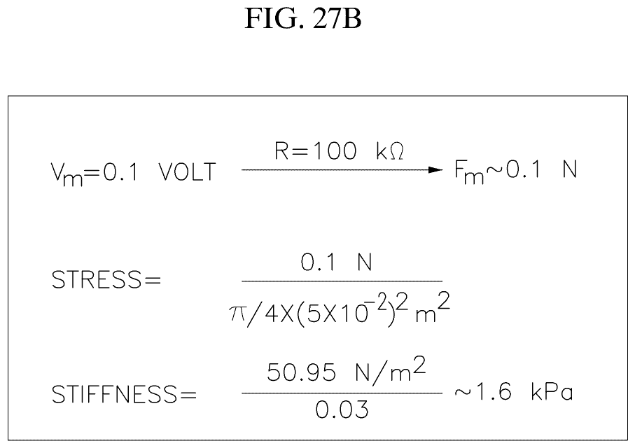

FIG. 27 shows stiffness measurement results of porcine lung using the compression-based device of the present invention. FIG. 27A is a schematic showing measurement of voltage and electrical current a via force sensor and contact electrodes, respectively, to determine the tissue stiffness. FIG. 27B is a table containing formulae for calculating tissue stiffness (i.e., modulus) using the measured electrical signals of FIG. 27A.

### EXAMPLE 6

### Alternative Compression-Based Palpation Device

A robotic tissue palpation device that can accurately, rapidly, and minimally invasively quantify tissue stiffness during robot-assisted minimally invasive surgery by determining elastic modulus (E) in situ (FIGS. 27A-27B) is disclosed. The device includes three components: i) a deployable sensing probe mounted on a steerable catheter that can compress local tissue for stiffness measurement; ii) a motion control module that enables multi-directional device movements, such as linear displacement, rotation, and deflection of the device; and iii) a micro-optical imaging module that provides the visual information during the probe navigation and tissue stiffness measurement.

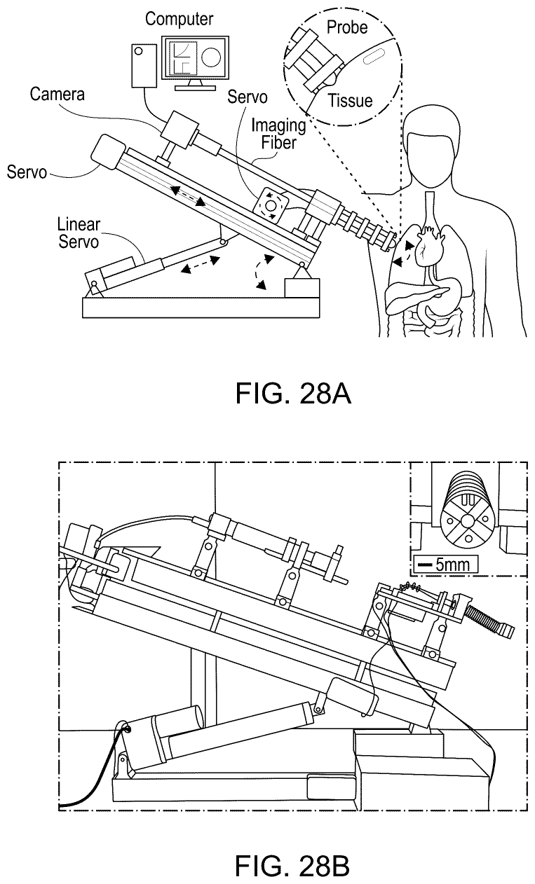

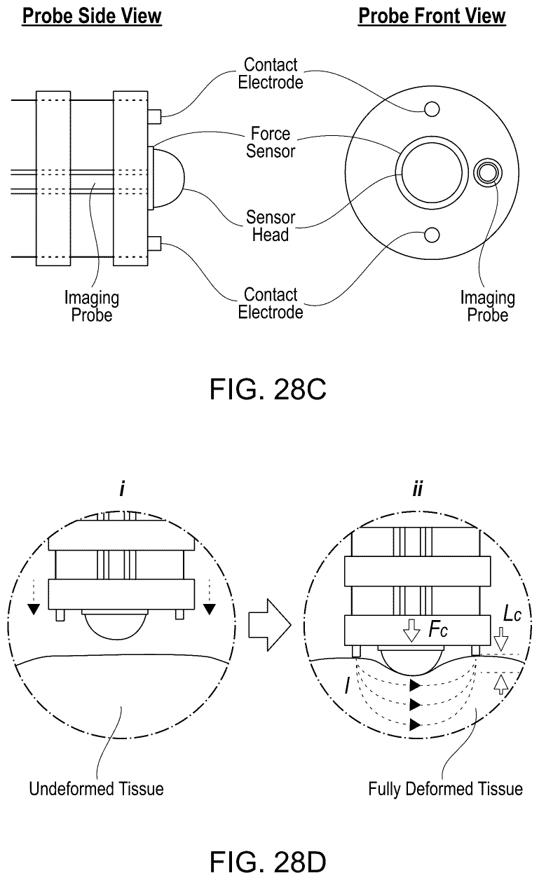

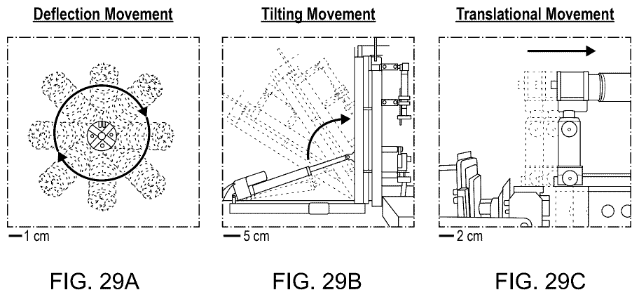

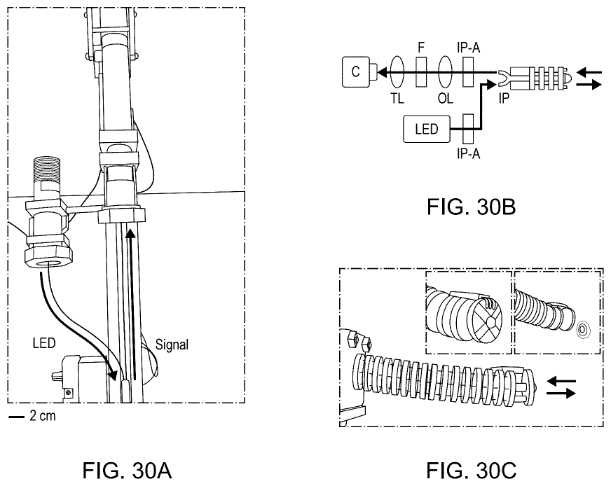

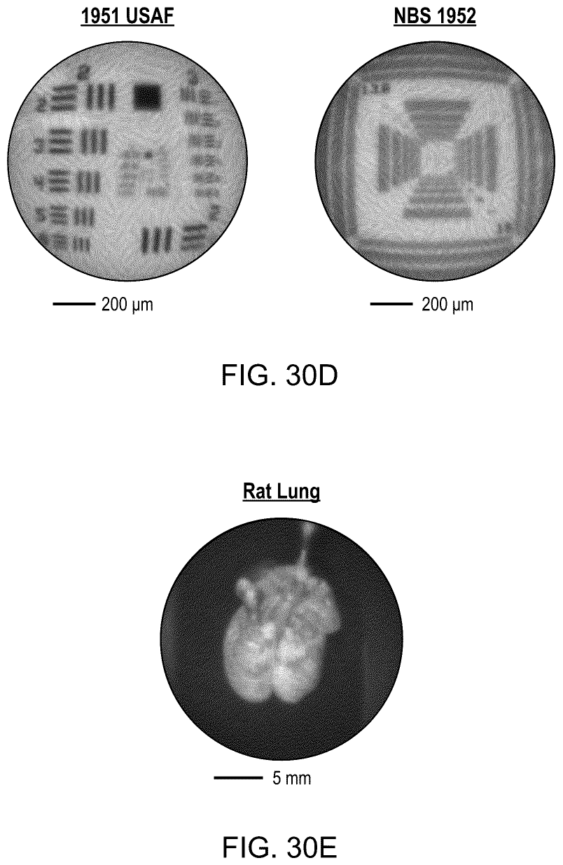

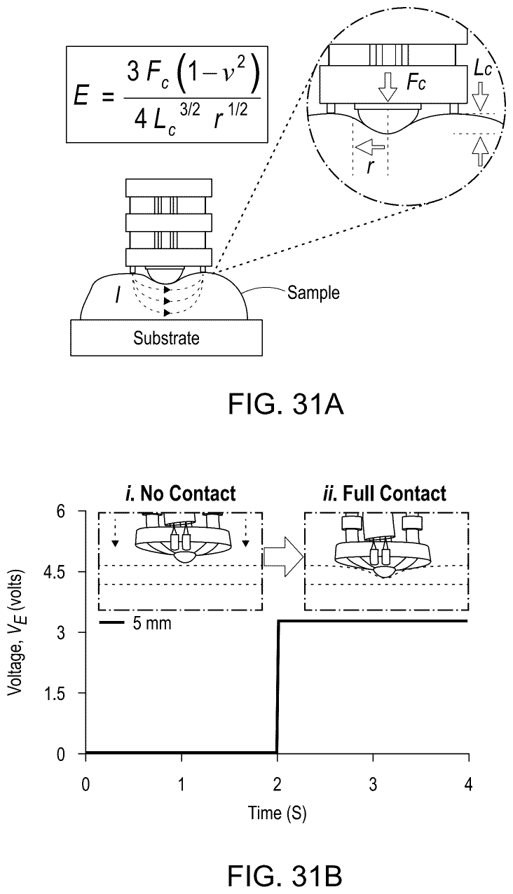

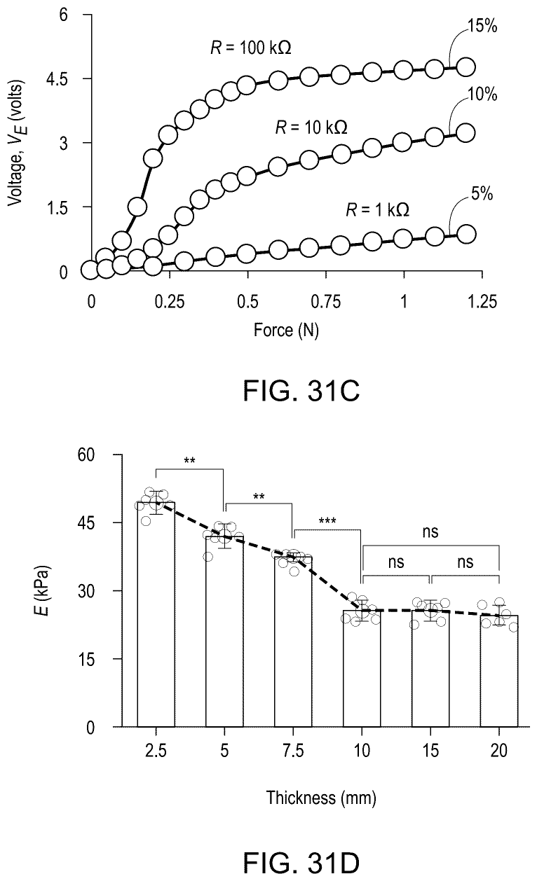

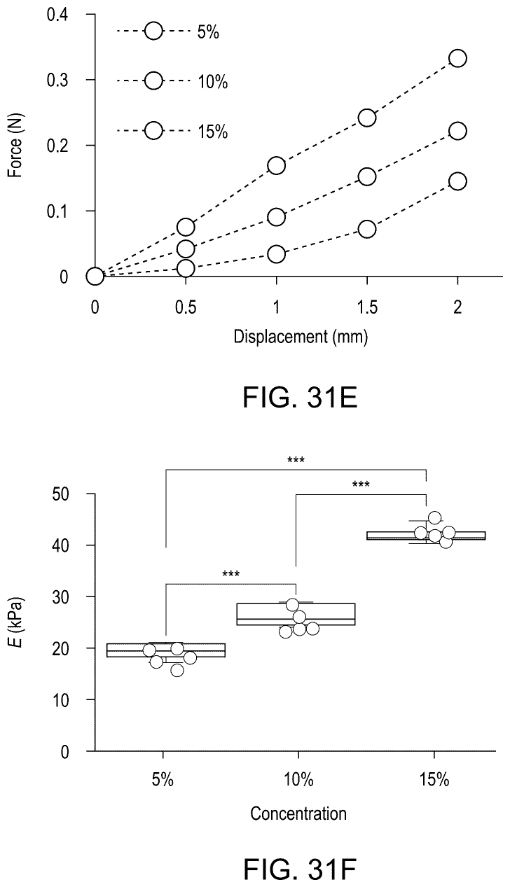

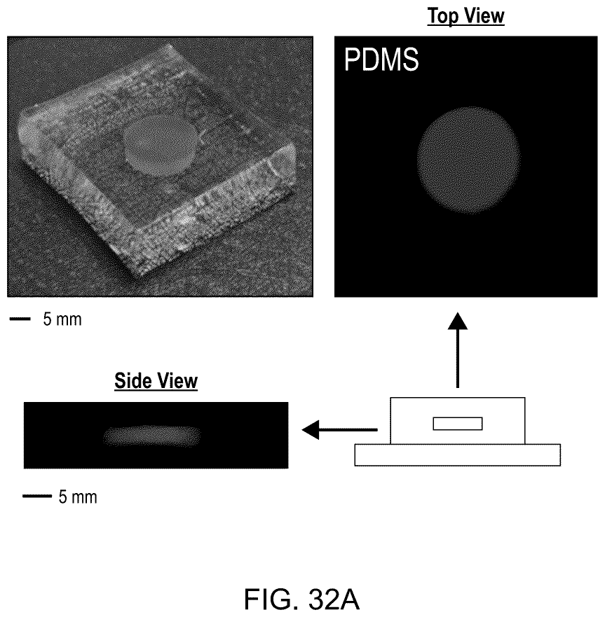

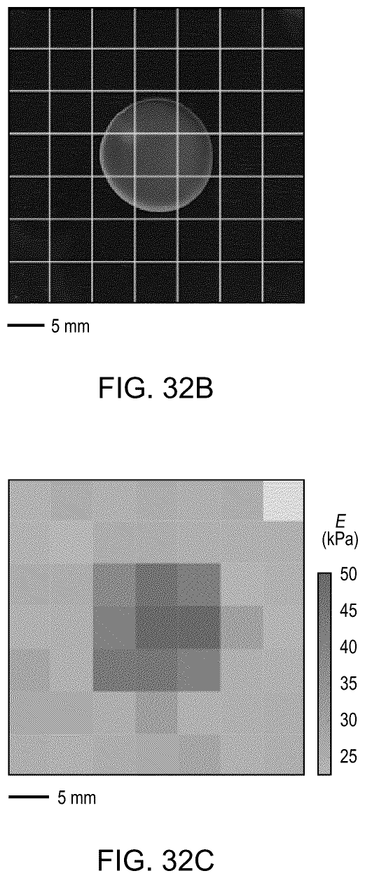

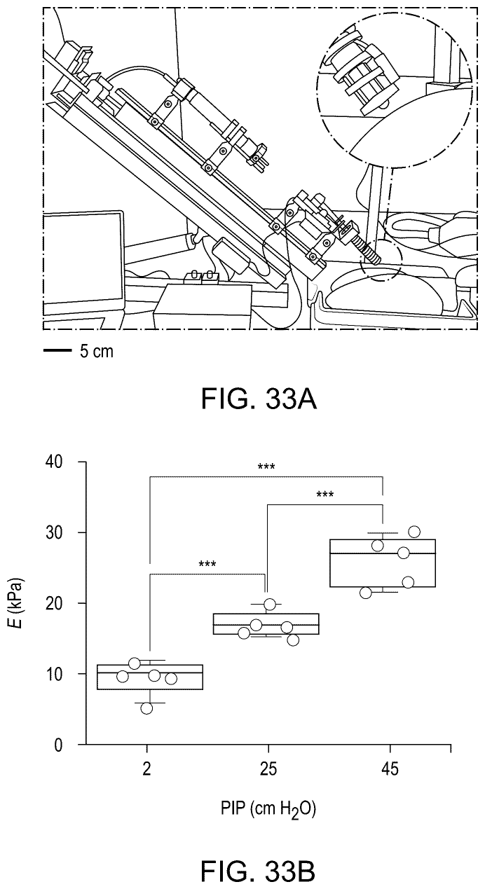

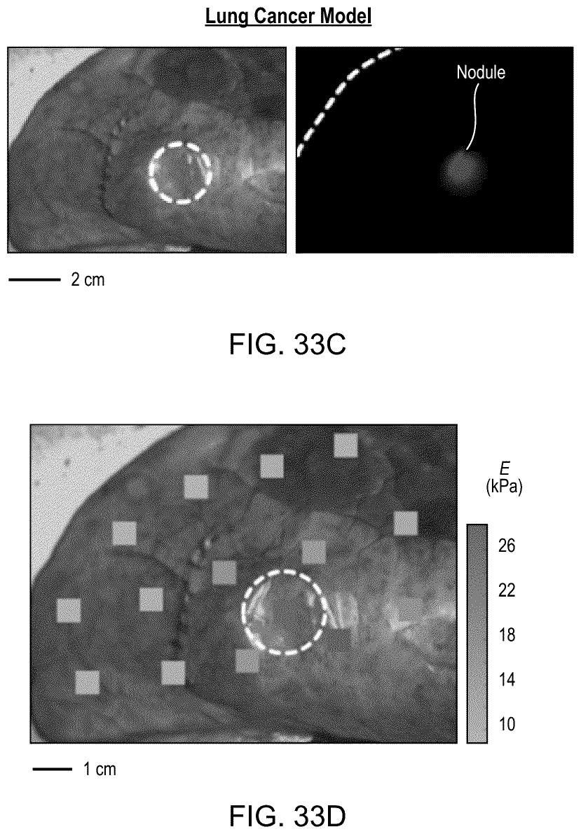

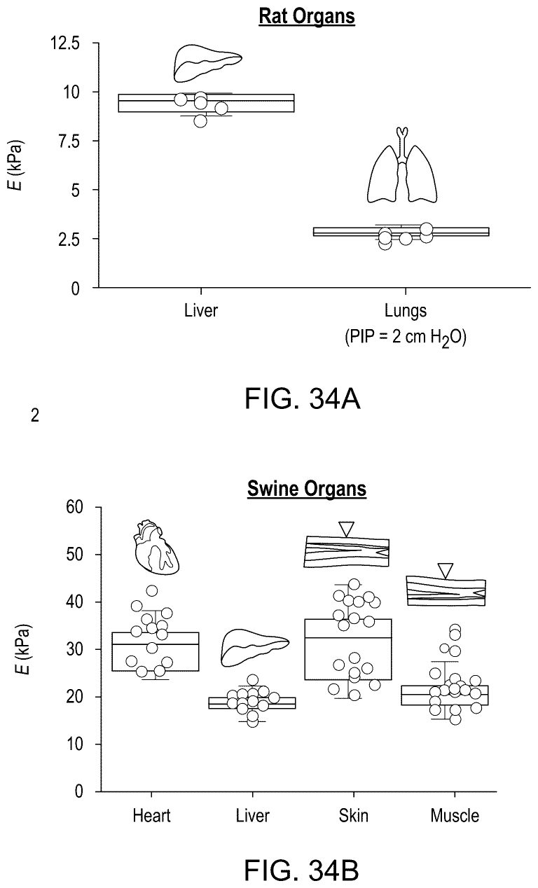

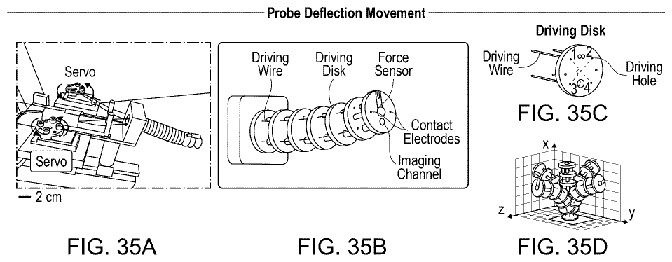

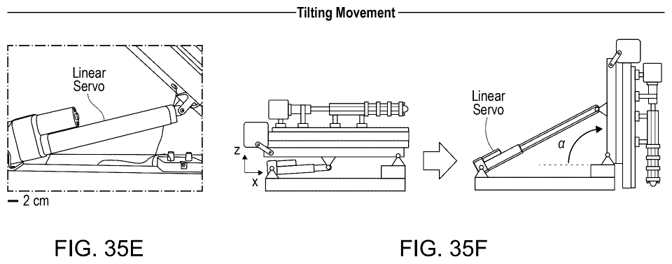

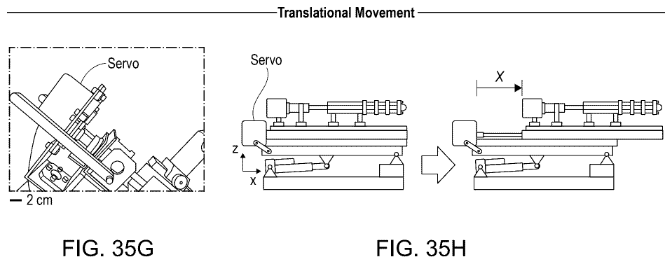

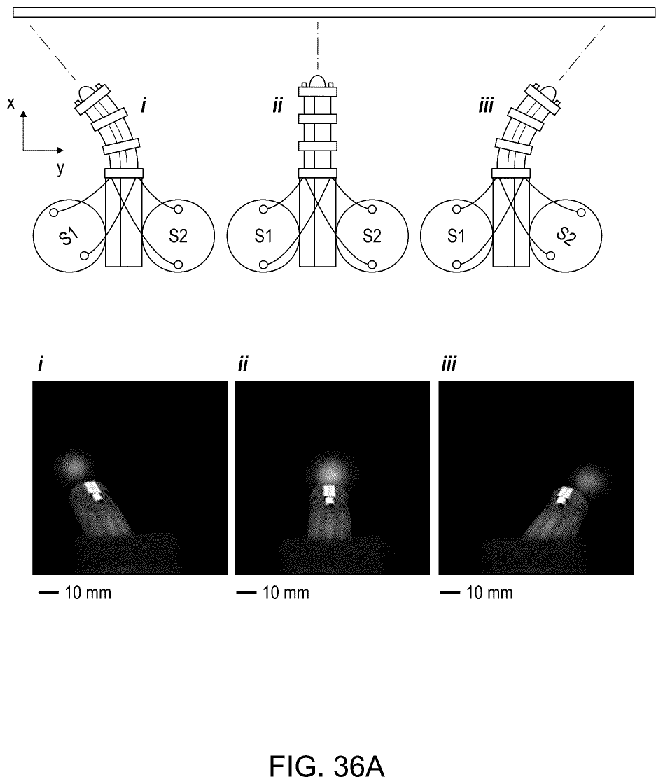

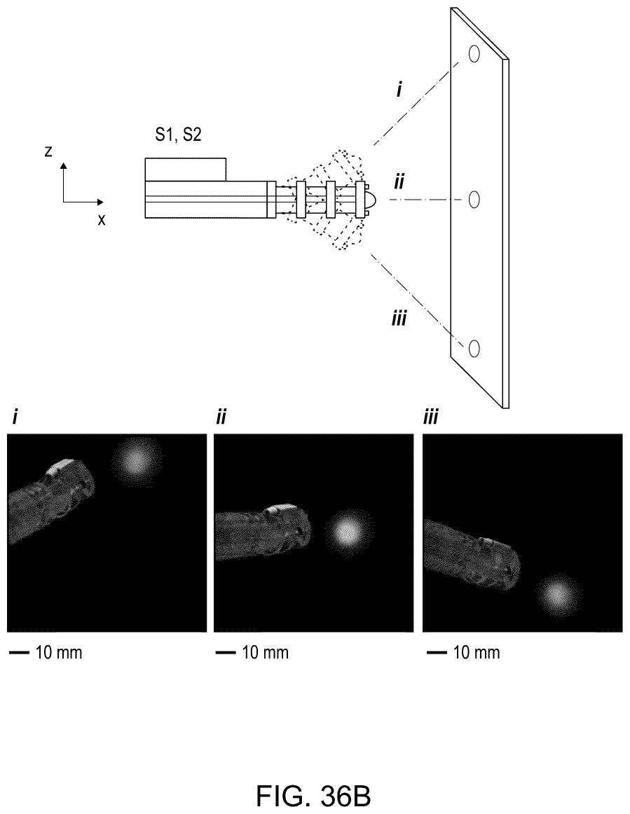

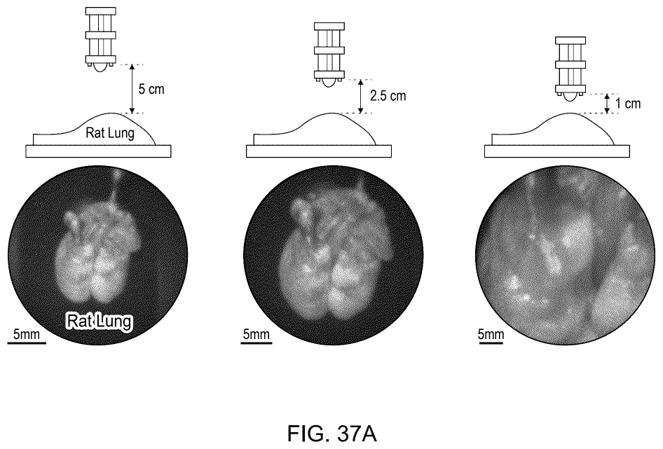

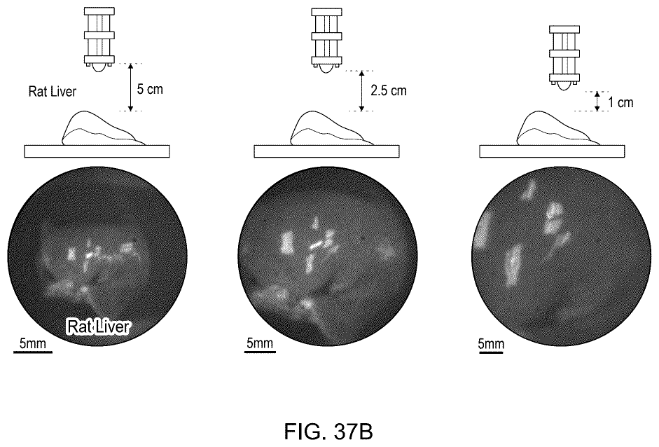

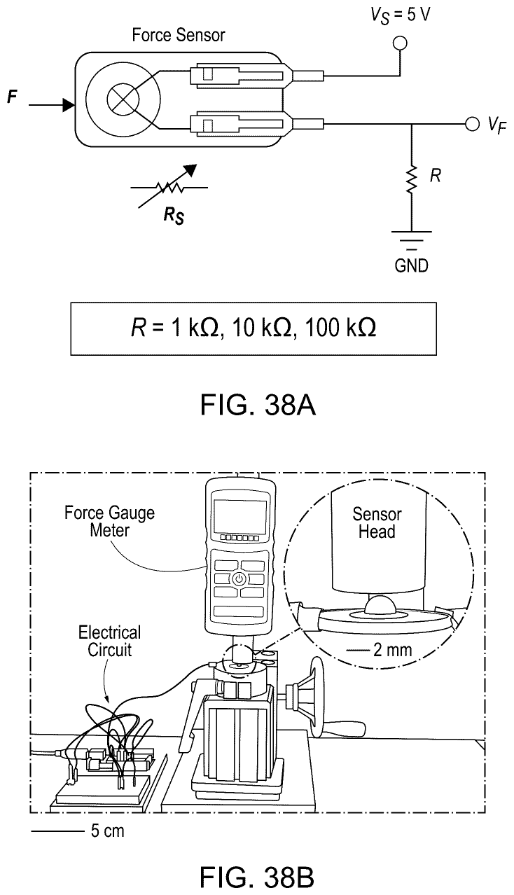

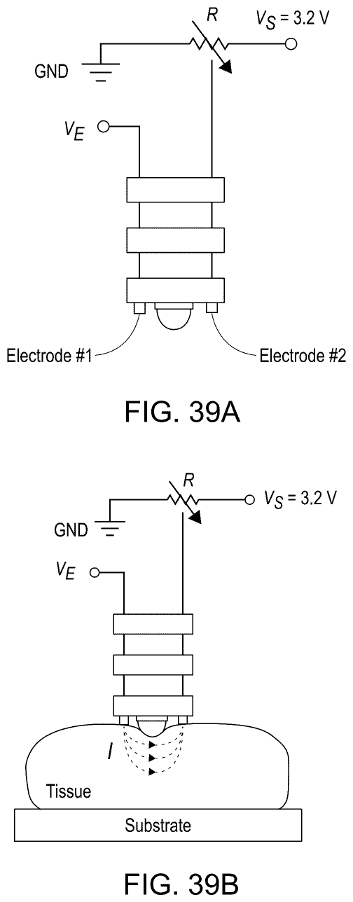

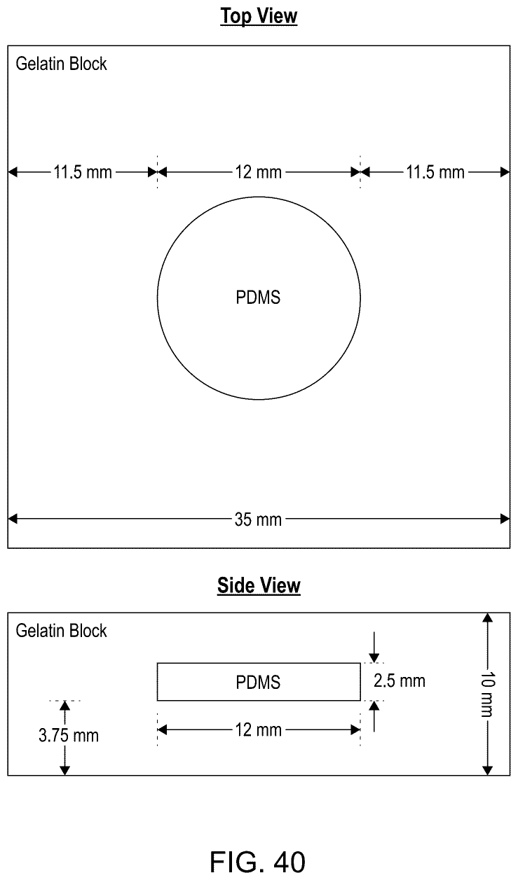

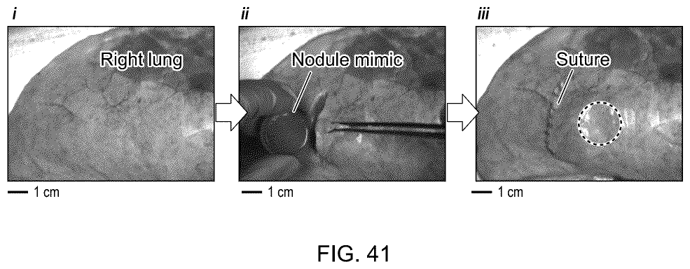

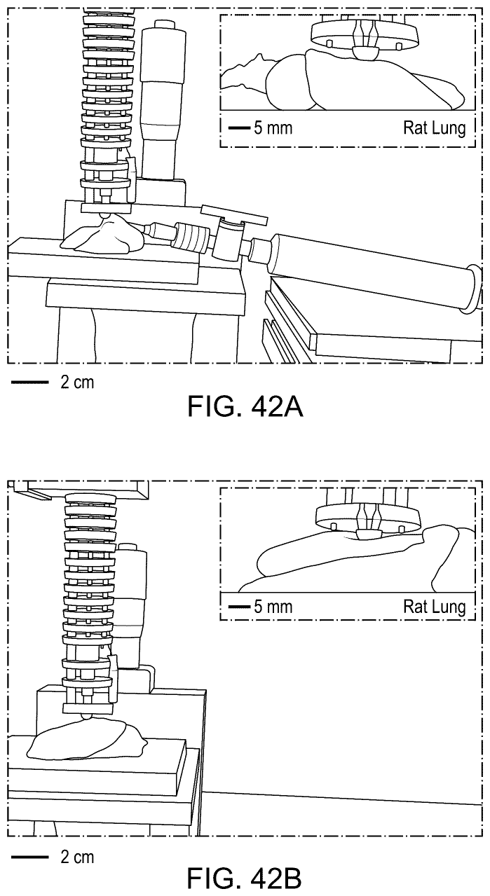

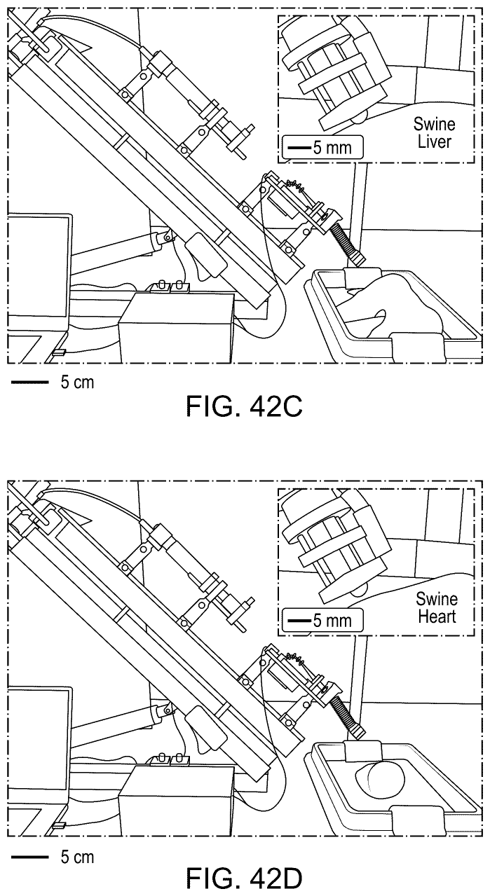

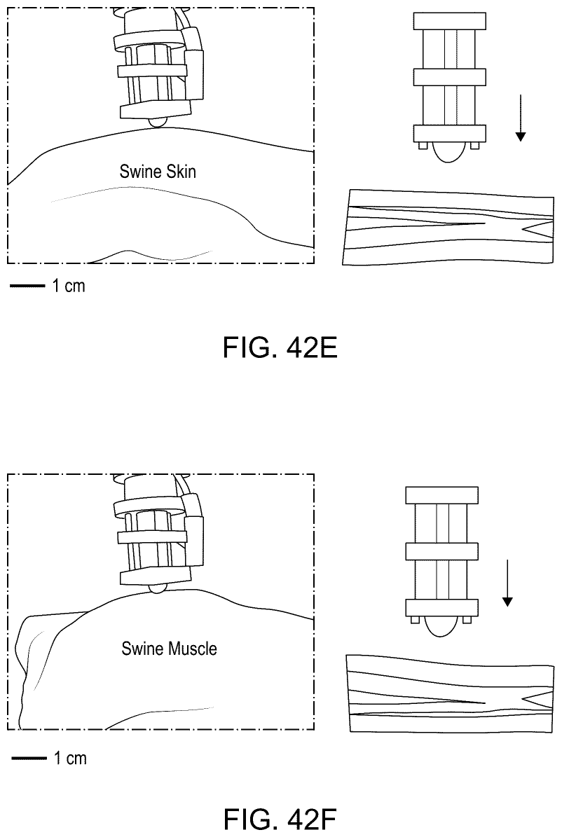

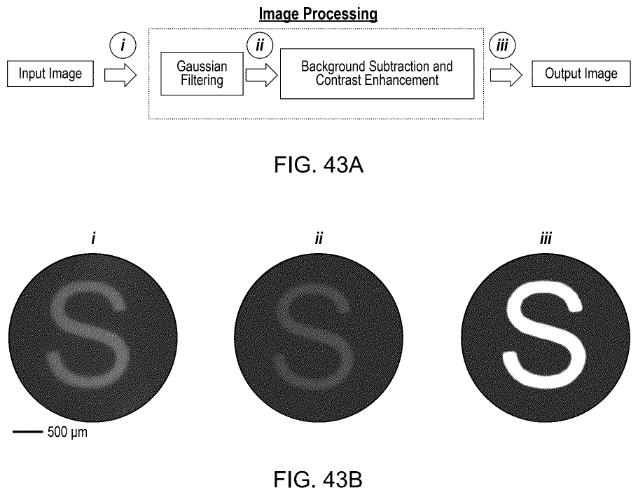

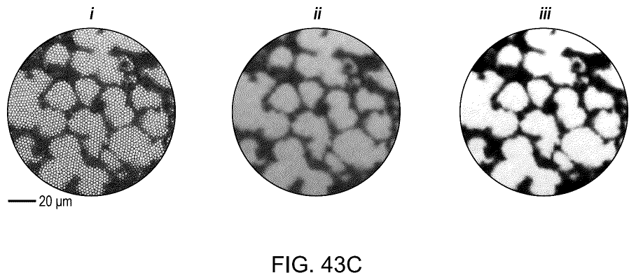

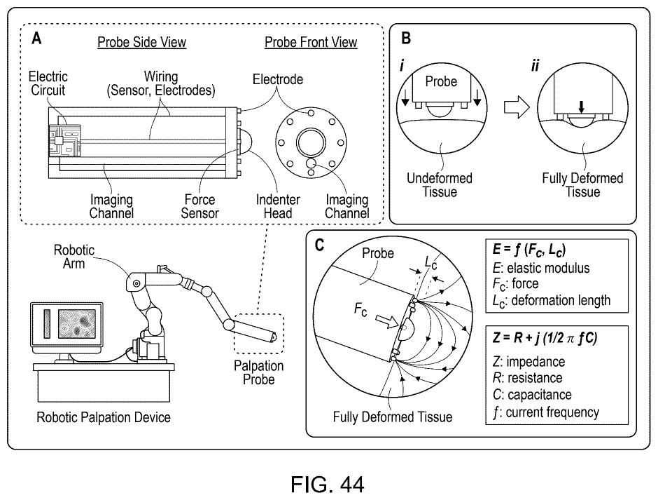

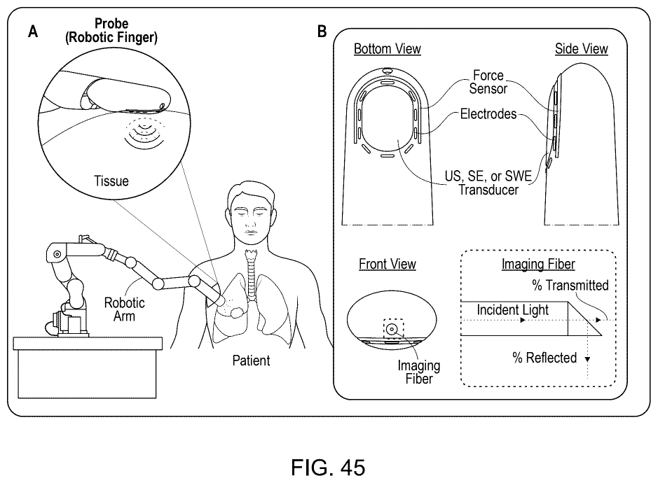

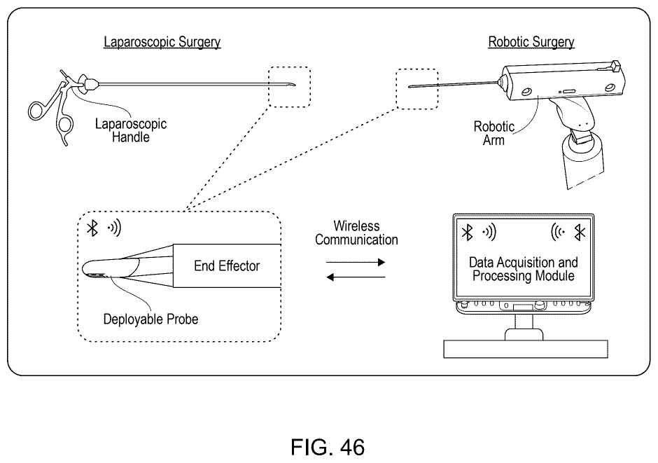

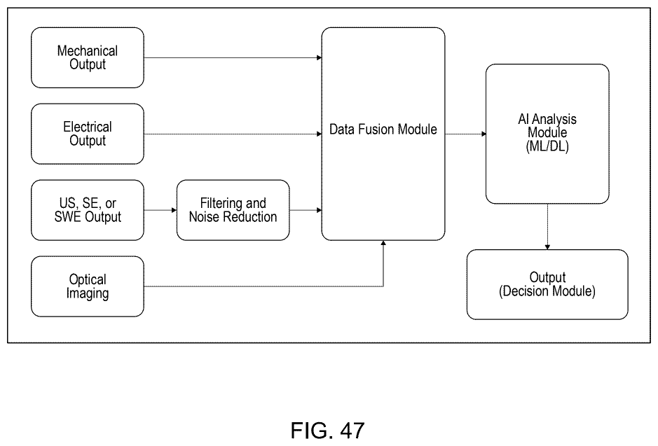

The stiffness measurement sensing probe includes a thin film-based force sensor (diameter: 3 mm), two contact electrodes (diameter: 1 mm), and a rigid hemispheric compression head made of acrylic plastic (height: 2 mm (FIG. 28C)). To utilize the force sensor in the stiffness measurement application, a force-to-voltage circuit is created using a digital data acquisition device, a reference resistor (R), and custom-written MATLAB code. In addition, an electrical circuit is formed between the contact electrodes (i.e., pogo pins) to quantify tissue deformation through acquisition of voltage as the readouts. To determine the tissue stiffness, the probe is directed downward perpendicular to the tissue surface (FIG. 28D, i). A force is then applied against the tissue surface via the hemispheric indenter to compress the tissue. Predefined magnitude of tissue deformation, which is determined by the height of the indenter (i.e., 2 mm), is confirmed when the electrical signal is detected across the two contact electrodes (FIG. 28D, ii). Then, the elastic modulus of the tissue is calculated by correlating the magnitude of the compression force (F_{c}) and deformation length of the tissue (L_{c}) using E=3F_{c} (1-2 v)/4L_{c}^{3/2} r^{1/2}, where E is elastic modulus, F_{c}: compression (indentation) force, v: Poisson ratio, L_{c} is deformation length, and r is the radius of sensor head.

FIGS. 35A-35H show the various motions of a robotic tissue palpation device. FIG. 36A is a photograph of the deflectable catheter that is controlled by two servo motors. FIG. 36B is a schematic showing the measurement probe and the wire-driven continuum robot design including driving disks and driving wires for multi-angular (deflection) movement of the catheter arm. FIG. 35C is a schematic of the structure of a driving disk and positions of driving wires on each disk. FIG. 35D is a schematic illustrating various deflection movements of the catheter arm in Cartesian coordinate system. FIG. 35E is a photograph of the linear servo that controls the tilting of the device. FIG. 35F is a schematic presenting that the extension of the linear servo arm results in tilting of the device with the angle a. FIG. 35G is a photograph of the servo motor that is responsible for linear displacement of the device probe. FIG. 35H is a schematic of the translational movement by the servo motor that results in a displacement X. FIGS. 36A and 36B are examples of deflection movement of the wire-driven catheter of FIGS. 35A-35H, showing movements in an x-y plane (35A) and an x-z plane (35B), wherein element ‘S’ is a servo motor.

The device functions to provide various movements, such as deflection, tilting, and linear displacement, to guide the measurement probe to the target tissue surface (FIGS. 29A-29C, 35A-35H, 36A-36B). The steerable catheter arm is designed as a wire-driven continuum robot and constructed with driving disks, driving wires, and flexible tubing. The robotic catheter is integrated into the tissue palpation device via a 3D printed adapter made of poly (lactic acid) (PLA) which is mounted on the device using dovetail rail carriers and a dovetail optical rail (FIG. 35A). The movement control module includes a linear motor that controls the tilting movement, a servo motor that modulates the translational movement, and two high-precision servo motors that control the deflection movements of the catheter's distal end. During the device's operation, all the motors are controlled simultaneously using two motor controllers. The deflection movement of the catheter arm is enabled by a wire-driven conformation that allowed multi-angular movements of the device tip in three-dimensional (3D) space (FIGS. 35A-35D, 36A-36B, 29A). The desirable probe deflection is achieved by pushing and pulling the driving wires via pulleys, mounted on servo motors. Tilting movement was enabled by extending or retracting a linear servo integrated into the device. Specific tilting motions of the device could be achieved by adjusting the length of the linear servo arm (FIGS. 35E-35F, 29B). Furthermore, a servo motor is installed on the back of the rail to control the linear motion of the measurement probe (FIGS. 35G-35H, 29C).

A custom-built imaging module is integrated into the tissue palpation device that allows vision-assisted navigation during device operation and tissue stiffness measurements (FIGS. 30A-30E, 37A-37B). The imaging module has an LED illumination light source, a flexible optical-fiber imaging bundle with embedded SELFOC micro-lens, a monochrome CMOS, an achromatic doublet, a 10x objective lens, a filter holder, a fiber bundle adapter, a translating lens mount, and extension tubes (FIGS. 30A, 30B). For imaging an object, such as an organ, the distal end of the imaging fiber bundle (i.e., imaging tip) is introduced into the imaging channel of the catheter, and the image formed on the proximal end passes through the objective lens and is collected by the camera's imaging sensor (FIG. 30C). Using this imaging module, bright-field images of imaging test targets (FIG. 30D) and the entire structure and smaller tissue regions of the ex vivo rat lungs (FIGS. 30E, 37A), and rat liver (FIG. 37B) are obtained.

To evaluate the functionality and measurement accuracy of the palpation device, the stiffness of the gelatin-based tissue phantoms was evaluated (FIGS. 31A-31F). The elastic modulus (E) was calculated using an equation developed for a spherical indenter (FIG. 31A). To determine the elastic modulus, the sensor head was gently pushed against the tissue phantom at a rate of 5 mm/min while the applied force was being recorded continuously (FIGS. 31B, 38A-38B). The tissue deformation continued until the tip of both contact electrodes touched the tissue surface due to maximum tissue deformation (i.e., 2 mm). The contact between tissue and electrodes was confirmed by detecting an electrical signal as the electrical circuit between the contact electrodes was closed (FIGS. 31B, 39A-39B). The voltage (V_{E}) recorded during the measurement showed a step response with 3.2 volts of the maximum voltage (FIG. 31B). The E values were then calculated based on the maximum deformation length (L_{c}: 2 mm) and the corresponding force measured (F_{C}).

The contact electrodes circuit is seen in FIGS. 39A and 39B. FIG. 39A is a schematic of the circuit and its components. FIG. 39B is a schematic showing the circuit of FIG. 39A touching tissue, wherein when the tips of contact electrodes touch the tissue, an output voltage (V_{E}) of 3.2 V is received, confirming the full contact as well as consistent tissue deformation. The labelled components are as follows: R: potentiometer resistance, VS: supply voltage, GND: ground, I: current.

Prior to all measurements, the force sensor circuit was calibrated by measuring the output voltages against known forces applied to the sensor (FIGS. 38A-B, 31C). As seen in FIG. 38A, the electrical circuit includes a force sensor, and a reference resistor (R). The input voltage (VS) is supplied by Arduino UNO (5 volts). The voltage between the of force sensor conductive layers (VF) in response to the applied force (F) is measured using custom MATLAB code. FIG. 38B is a photograph illustrating the calibration of the force sensor. Known force loads are applied to the sensor head (indenter) by the force gauge meter, and the received voltage is measured by the electrical circuit. For FIG. 38A the elements are as follows: F: force, R: reference resistor, RS: the resistance between force sensor conductive layers, GND: ground, VS: supply voltage.

Different reference resistors (R; 1, 10, and 100 kΩ) integrated into the sensor circuit were tested to investigate the role of the resistance on the measurement outcomes (FIGS. 31C, 38A-38B). The output voltage (VF) versus force load (F) curves were generated for all resistors (force range: 0-5N) (FIG. 31C). Results showed that increasing the value of reference resistance increased the sensitivity of the force measurement at lower force ranges (i.e., below 0.5 N). Since lung and liver tissues are soft, the 100 kΩ-resistor was used for the stiffness measurements.

Next, the stiffness of gelatin tissue phantoms (concentration: 10% w/v) of different thicknesses (2.5, 5, 7.5, 10, 15, and 20 mm) was evaluated to find the critical tissue thickness at which the effect of the substrate is negligible (FIG. 31D). The measured E values were 49.5±2.6, 42.1±2.8, 37.4±0.7, 25.7±2.3, 25.7±2.2, and 24.5±2.3 kPa for phantoms with the thickness of 2.5, 5, 7.5, 10, 15, and 20 mm, respectively. These results show that the effect of the substrate on the measured stiffness decreased as the phantom thickness increased, without significant difference in stiffness for tissue phantoms thicker than 10 mm (FIG. 31D).

Further, the stiffness of phantoms with different gelatin concentrations (5, 10, and 15% w/v; phantom thickness: 10 mm; FIGS. 31E, 31F) was measured. The phantoms were compressed using the device while the deformation was continuously monitored using a camera. The slope of the acquired force-displacement curves increased with the gelatin concentration (FIG. 31E). For 5, 10, and 15% w/v tissue phantoms, the measured E was 18.3±1.5, 25.7±2.2, and 42.5±1.6 kPa, respectively (FIG. 31F). The results were in the range of elastic moduli of physically crosslinked gelatin phantoms measured via the vacuum-based method described herein.

Furthermore, tissue phantoms were used to investigate whether the palpation device could profile tissues with spatially heterogenous stiffness, such as a tumor formed in tissue (FIG. 32A-32C). To this end, an ICG-labeled cylindrical-shaped PDMS (or, alternatively, silicone: e,g, ratio of prepolymer to crosslinker: 10:1; diameter: 12 mm; thickness: 2.5 mm)), which recapitulated a stiff nodule, was embedded at the central region of a soft 10% gelatin block (e.g., concentration: 10% w/v; length: 35 mm; width: 35 mm, thickness 10 mm; FIGS. 32A, 40). The block can be prepared by dissolving gelatin powder (G2500, Sigma-Aldrich) in 1xphosphate buffered saline (1xPBS; Gibco) at 70° C. and physically crosslinking the solutions at 4°° C. for 30 minutes. The location of the ICG-labeled PDMS nodule was confirmed visually via NIR imaging (FIG. 32A). Then, the stiffness of the tissue phantom was mapped using the palpation device (FIG. 32B). Starting from the upper left corner of the phantom, the stiffness was measured by gently compressing the surface of the tissue mimic and recording the force and electrical signals. The stiffness map showing the elastic modulus distribution with a spatial resolution of 5 mm was generated (FIG. 32C). Notably, while the E values at peripheral areas of the phantom block varied between 23 to 27 kPa, there was a substantial increase in the E values (range: 38 to 50 kPa) at the center of the tissue phantom where the PDMS-based (or silicone-bsed) nodule was located, highlighting the ability of the device in accurate and quantitative localization of a stiff tissue mimic.

It was also investigated whether the palpation device could accurately measure the elastic modulus of human-sized swine lungs that were subjected to various internal air pressures (FIGS. 33A-33B). The tissue stiffness was measured without ventilating the lungs to minimize motion-induced measurement errors. Specifically, the intra-peak inspiratory pressure (PIP) of the lungs were adjusted to a constant level (e.g., 2, 25, and 45 cmH₂O) during each measurement using an Ambu bag and a pressure sensor integrated into the tracheal tubing (FIG. 33A). The E values were measured to be 9.1±2.3, 16.8±1.8, and 26.0±3.6 kPa when the internal pressure of the lungs (PIP) was subjected to 2, 25, and 45 cmH₂O, respectively (FIG. 33B). Notably, the lung tissue stiffness increased with PIP due to changes in the tension (T) of the lung parenchymal tissue within the pleural network and alveolar septa with respect to the pressure inside the lungs. Additionally, the E values measured using the device were in the range reported in the literature.

Further, a lung tumor model was created that mimicked the presence of small nodules (size: 2 cm) in the distal regions of the lungs to determine whether the palpation device could accurately discriminate the nodules from healthy tissue (FIGS. 33C-33D). The lung cancer model was generated using an explanted swine lung and an ICG-labelled PDMS block as a nodule mimic (diameter: 2 cm) (FIG. 33C). An incision was created in the right lobe, and the nodule was placed in the subpleural region, approximately 5 mm below the pleura surface (FIGS. 33C, 41). The incised area can be sutured (4-0 silk) to prevent air leak and lung collapse. The imaging module of the palpation device was used to visualize the location of the nodule in the subpleural region (FIG. 33C). The measurement probe was then moved to different regions of the lobe (distance between measured regions: ˜1 cm) to evaluate the stiffness across the lung tissue near the region where the nodule mimic was implanted. Following the scanning, a stiffness map showing the distribution of the elastic modulus across the entire lobe was generated (FIG. 33D). Notably, the E values ranged from 9.3 to 28.3 kPa, where the stiffness of 28.3 kPa corresponded to the precise location of the nodules in the lung lobe, confirming the ability of the device to locate the small nodule in the lung.

The tissue stiffness of rat liver and lung, and swine heart, liver, skin, and muscle (FIGS. 34A-34B, 42A-42F) was investigated. The measured E values were 2.6±0.3 and 9.2±0.5 kPa for rat lung (peak inspiratory pressure: 2 cmH₂O) and liver, respectively (FIG. 42A). The measured E values were 33.0±5.4, 19.2±2.2, 33.5±8.2, and 22.6±6.0 kPa, for swine heart, liver, abdominal skin, and muscle, respectively, and were similar to the values reported in the literature.

To remove the core boundaries of the fiber bundle and smoothen the images taken by the imaging probe of the palpation device, the Accurate Gaussian Blur filter technique (FIG. 43A-43C) was used. The parameter of decay (Sigma; radius) was adjusted to 4. The background was removed, and the contrast and brightness of the images were adjusted.

The present device improves over prior devices with respect to intraoperative minimally invasive and localized in situ stiffness measurements. The closest technologies to the present robotic palpation device are ultrasound-based technologies/devices, such as FibroScan and Elastography. They are commonly used to assess the degree of liver fibrosis in patients with chronic liver diseases such as hepatitis, alcoholic liver disease, or non-alcoholic fatty liver diseases, and breast lesions. While they offer assessment of tissue stiffness for solid organs, such as breast and liver, their bulky probes make them impractical for robotic surgery. Moreover, their application is limited to specific tissues and organs, and they are susceptible to frequent measurement errors.

The present palpation device also facilitates accurate localization of lung nodules during robot-assisted surgery. Currently, there is no gold-standard method for identifying small pulmonary nodules in the lungs. Current technologies, such as pre-operative localization with wires, markers, and dye are limited in their effectiveness and applicability. Further, existing pre-operative image-guided modalities, such as MRI and PET are not favorable for the accurate localization of nodules due to the mismatch between the inflated lung in pre-operative images and the partially/fully deflated lung during the surgery. The present robotic tissue palpation device, however, can pinpoint the small nodules in the lungs in real time and allow surgeons to make objective and data-driven decisions during resection procedures.

The present device also enables an electrical-based accurate determination of tissue deformation. The current deformation measurement methods, such as optically based and computer-assisted approaches, are time-consuming and prone to errors. For accurate quantification of tissue stiffness, one can determine the exact deformed shape of the tissue caused by tissue compression. In the present device, the maximum tissue deformation (endpoint of the stiffness measurements) can be accurately determined in real-time via two contact electrodes embedded on the tip of the probe. This detection method allows accurate measurement of the maximum tissue deformation from which the stiffness of the tissue being evaluated can be calculated. Additionally, after contacting the tissue surface, the electrodes can measure the electrical properties, i.e. electrical resistance, of the tissue of interest.

As previously discussed, the present device can be integrated with a fiber-optic-based imaging probe module that allows visual inspection during device navigation and stiffness measurements. The imaging module can be modified for both tissue-and cellular-level imaging. In addition, the flexibility of the imaging fiber facilitates integration into the steerable catheter that is inserted in the surgery port. Moreover, the bifurcated geometry of the imaging fiber enables simultaneous illumination and imaging, with the light passing through the “transmitting bundle” to the fiber tip and the fluorescent signal passing through the “receiving bundle” into the camera. This imaging capability can be useful during intra-operative tumor resection, where surgeons can administer fluorescent molecules (e.g., fluoresceine, rhodamine, tagged antibodies, Indocyanine Green) that can specifically label tumors to improve the accuracy of tumor identification and resection.

With these features, the present inventive device addresses the lack of commercial devices to intraoperatively detect diseased tissues with altered stiffness in robot-assisted surgeries. The invention also addresses the lack of commercial devices to measure mechanical properties, in particular, elastic modulus, of soft tissues, organs, and biomaterials without compromising their native structure.

### Detailed Methods

The catheter (diameter: 2 cm, length: 10 cm) was comprised of a wire-driven arm and a stiffness measurement probe (FIGS. 35A-35H). The catheter arm was made of driving disks (diameter: 2 cm, thickness: 2 mm, spacing between disks: 1 mm) that were cut from a transparent acrylic plastic sheet (McMaster-Carr) using a laser cutter (Full Spectrum Laser), driving wires made of nylon (diameter: 0.75 mm, McMaster-Carr), and flexible tubing (inner diameter: 1 mm, outer diameter: 2 mm, McMaster-Carr). Nine holes were drilled in each driving disk: four holes that were evenly distributed around the disk for guiding the driving wires (diameter: 2 mm, angular distance: ) 90°, one hole for introduction of flexible tubing as an imaging channel (diameter: 2 mm), and 4 holes for accommodating the contact electrode and force sensor wires (diameter: 1.5 mm). The stiffness measurement probe was constructed by attaching a force sensor (diameter: 3 mm; GD03-10N, UNEO), two spiral-headed pogo pins as contact electrodes (diameter: 1 mm, MilMax), and a rigid hemispherical head made of acrylic plastic onto an acrylic disk (diameter: 2 cm, thickness: 2 mm) on the distal end of the catheter arm. The whole catheter was integrated into the device through a 3D printed platform made of poly (lactic acid) (PLA; MakerBot), which was mounted on the device arm using dovetail rail carriers (RC1, Thorlabs) and a dovetail optical rail (RLA0600, Thorlabs).

To integrate the force sensor into the stiffness measurement application, a force-to-voltage circuit was created using a digital data acquisition device (Arduino UNO, Rev 3), a reference resistor (R, Microchip), and custom-written MATLAB code (MATLAB R2021) (FIGS. 38A-38B). The reference resistor was incorporated into the circuit in a voltage divider configuration by which the output voltage (VF) increases with respect to the added force. The force sensor was calibrated by applying a known force load using a force gauge device (M5-50, Mark-10) and measuring the output voltage received by the sensor. To ensure accurate force reading, the force was evenly distributed across the sensing area using a platen attached to the force gauge device. Different reference resistors (1 kW, 10 W, and 100 KW) were tested to generate various voltage-force plots and determine the sensitivity of the force measurement circuit. The 100 KW resistor was selected for further stiffness measurements based on the circuit's sensitivity in response to the applied force.

An electrical circuit was created between two contact electrodes to confirm the probe-tissue full contact and to create a consistent deformation on tissues (FIGS. 39A-39B). The circuit was made using an Arduino board (UNO, Rev3) and a potentiometer (Microchip) to measure the voltage between the contact electrodes. The circuit output (V_{E}) shows a stepwise increase in voltage (i.e., 3.2 volts) when a current is established between the electrodes confirming the maximum deformation.

Lungs and liver were harvested from Sprague-Dawley rats (SAS SD rats; total: 12 rats; weight: 250-270 g; Charles River Laboratories). All animal care, handling, and experimental work were conducted under the animal protocol approved by the Stevens Institute of Technology Institutional Animal Care and Use Committee (IACUC). In addition, all methods complied with the National Institutes of Health (NIH) Guide for the Care and Use of Laboratory Animals, eighth edition. To isolate the lungs and liver, a rat was euthanized through inhalation of 2.5% isoflurane in the air for 15 min in an induction chamber using a small vaporizer (Kent Scientific). The depth of anesthesia was confirmed by evaluating the pedal reflex (i.e., pinching the

animal's foot pad). Subsequently, 1 mL of 1,000 units/mL heparin was injected through the lateral vein to prevent blood clotting in the pulmonary vasculature, and the animal was euthanized via inhalation of 5% isoflurane in the air for an additional 15 min. The animal was then fixed on a surgical board by immobilizing the legs and tail.

To harvest the liver, an incision was made in the abdominal cavity, and the entire liver was dissected carefully from the surrounding connective tissues. The lungs were harvested via a tracheotomy procedure that involved making an incision in the neck midline and exposing the trachea, and connecting a Luer connector (diameter: 2 mm, Harvard Apparatus) to the trachea using a silk suture (size: 4). The lungs were then partially inflated by injecting 1 mL of air through a 10 mL syringe. Next, an incision was made in the chest wall by cutting the ribs, and the lungs and heart were isolated from the chest cavity by cutting the surrounding connective tissues. After harvesting, the liver and lungs were rinsed with saline solution (1′PBS) prior to the

stiffness measurements. Lungs were mechanically ventilated (e.g., with a manual resuscitator, such as an Ambu bag) for 10 min, and connected to a pressure sensor, with a tidal volume of 2.2 mL to eliminate the variation between the lungs that could be caused by inflation and deflation history.

Poly (dimethylsiloxane) (PDMS) mixtures were made by mixing pre-polymer base and crosslinking curing agent at the ratio of 10:1 (SYLGARD™ 184 silicone elastomer kit, Sigma). To label the PDMS for near-infrared imaging, indocyanine green (ICG) fluorescent dye (excitation/emission: 785 nm/830 nm) solution in glycerol (concentration: 1 mg/mL) was added to the PDMS mixture prior to crosslinking. Next, trapped air bubbles were removed from the mixture using a vacuum pump. Finally, the ICG-labelled PDMS samples were fabricated by curing the mixture in a dark environment at room temperature for 48 h.

To create tissue phantoms with heterogenous stiffness, a cylindrical PDMS (component with greater stiffness) was embedded at the center of a gelatin block with a concentration of 10% w/v (component with lower stiffness). The gelatin solution was poured into a cubic silicon mold (35 mm×35 mm) to create a hydrogel layer with a thickness of 3.75 mm. After crosslinking, the layer at 4° C., PDMS (diameter: 12 mm, thickness: 2.5 mm) was placed at the center of the mold on the top of the gelatin layer. Fresh gelatin solution was added to cover the PDMS nodule and the bottom gelatin layer to generate a phantom block with a final thickness of 10 mm.

To calculate the resolution of the images obtained by the imaging system using the USAF 1951 target, the following equation was used:

$$Resolution ( \frac{line pair}{mm} ) = {2}^{Group + ( \frac{Element - 1}{6} )} \qquad [ S 1 ]$$

Where Group is the number of smallest visible group of line sets, and Element is the smallest visible line pairs (one light line and one dark line) in the target. In the images taken by the imaging system, the smallest visible group was 3, and the element was 5 when the working distance was set to 1 mm. Therefore, the resolution was determined to be 80 mm/line pair (one light line and one dark line) using the equation immediately above. In order to calculate the resolution of the images with the NBS 1951 test target, the following table (Thorlabs) was used:

| | Resolution (line/mm) | | | | | | | | | | | |
|---|---|---|---|---|---|---|---|---|---|---|---|---|
| | 4 | 4.8 | 5.6 | 6.8 | 8 | 9.6 | 11.2 | 12 | 13.6 | 14 | 16 | 17 |
| Line | 125 | 104.2 | 89.3 | 73.5 | 62.5 | 52.1 | 44.6 | 41.7 | 36.8 | 35s.7 | 31.3 | 29.4 |
| width | | | | | | | | | | | | |
| (mm) | | | | | | | | | | | | |

The resolution of the images was approximately ˜74 mm/line pair using NBS 1951 test target.

To calculate the elastic modulus of soft tissues using the indentation method, the theoretical model for rigid spherical indenters was used as follows [S1-S3]:

$${E}_{eff} = \frac{3}{4} \frac{{F}_{c}}{{L}_{c}^{3 / 2} * {r}^{1 / 2}} \qquad [ S 2 ]$$

Where E_{eff} is the effective elastic modulus, F_{c} is the indentation force applied by the indenter, Lₑ is the indentation depth (deformation length), and r is the radius of the spherical indenter. Equation [S2] can successfully approximate the elastic modulus of the tissue at low deformation lengths [S3]. E_{eff} is defined as:

$$\frac{1}{{E}_{eff}} = \frac{1 - {v}_{s}^{2}}{{E}_{s}} + \frac{1 - {v}_{i}^{2}}{{E}_{i}} \qquad [ S 3 ]$$

Where Eᵢ, nᵢ and Eₛ, nₛ are Young's modulus and Poisson ratio of the indenter and the tissue, respectively. It is assumed that the indenter is infinitely rigid (Eᵢ>>>Eₛ); thus, Equation [S3] can be re-written as:

$$\frac{1}{{E}_{eff}} = \frac{1 - {v}_{s}^{2}}{{E}_{s}} \qquad [ S 4 ]$$

By plugging Equation [S4] in Equation [S2], the elastic modulus of the tissue can be determined by the following equation:

$${E}_{s} = \frac{3}{4} \frac{{F}_{c} * ( \frac{1 - {v}_{s}^{2}}{{E}_{s}} )}{{L}_{c}^{3 / 2} * {r}^{1 / 2}} \qquad [ S 5 ]$$

The indention depth at maximum deformation (i.e., the height of hemispherical head; the distance between the tips of contact electrodes and the tip of the hemispherical head; L_{C}=2 mm) was used to calculate the elastic modulus. The radius of the hemisphere head was 2.5 mm. The Poisson ratio was assumed to be 0.5 for the tissue phantoms and the biological tissues [4-7].In addition, the stiffness quantification of tissue was based on the linear elasticity assumptions of the tissue phantoms and biological tissues. In order to maintain the linear elasticity, the tissue deformation was maintained lower than 2 mm, and the measurements were performed at a low strain rate (5 mm/min).

To detect the ICG fluorescent signals of PDMS cylinders embedded in tissue phantoms and swine lungs, a custom-built NIR imaging setup was constructed. The NIR imaging system was comprised of a scientific CMOS camera (Manta G-145 NIR, Allied Vision), a camera lens (50 mm C-Series VIS-NIR, Edmund Optics), a 785-nm laser device (MDL-III-785, OptoEngine), and NIR filter (ICG-B-000, Semrock). The exposure time of images was adjusted using Vimba Viewer software (Allied Vision) to obtain optimal NIR images. The image processing procedure was performed to produce multiplicity images showcasing the device's various motions, including catheter deflection, translational, and tilting movements. To generate a multiplicity image, first, a sequence of time-lapsed photos was captured during each movement using a fixed camera. The photos were then imported as a stack into a raster graphics editor (Photoshop, version: 22.1). The photo with the lowest time index (t=0) was selected as the background photo. Next, a mask was generated on each subsequent clone photo (t>0) using the mask tool in the layer window. Starting with the first clone, the unwanted areas of each clone were carefully erased on the mask layer using the brush tool (size: optional, hardness: 0, opacity: 100%, flow: 100%). Finally, the final multiplicity photo was generated by saving all layers into a single tiff format image.

To remove the core boundaries of the fiber bundle and smoothen the images in FIG. 43, the Accurate Gaussian Blur filter plugin in FIJI (ImageJ2) software was used. The parameter of decay (Sigma; radius) was adjusted to 4. The background was removed using the Process>Math>Subtraction option. Finally, the contrast and brightness of the images were adjusted using the Adjust>Brightness/Contrast option.

To measure the elastic modulus of PDMS (the ratio of pre-polymer to crosslinker: 10:1), compression testing was performed according to the D965-15 ASTM standard via an Instron machine (5965, load cell: 5 kN) at the crosshead speed of 1 mm/min for one cycle. The elastic moduli were determined by calculating the slope of the linear region of the stress-strain curve. As a result, the elastic modulus of PDMS was measured to be 233.3±16 kPa.

### EXAMPLE 7

### Electrical Properties Measurement

In a further embodiment of the present invention, the device further measures the electrical properties (i.e, conductivity, impedivity, permittivity, etc.) of the tissue of interest along with mechanical measurements (FIGS. 44A-44C). The probe gently deforms the tissue to measure the mechanical and electrical properties (FIG IB). Mechanical measurements are performed using an indenter and a force sensor, while contact electrodes enable electrical measurements. In this approach, the probe is equipped with, in addition to an array of contact electrodes, electrical circuit and electrical sensors that inject an electrical current into the tissue of interest and simultaneously measure the resulting electrical properties of the tissue. These electrical tissue assessments can provide insights into the tissue's biological composition and structural integrity, complementing the mechanical assessments. The electrical measurements can allow distinguishing between healthy and pathological tissue by identifying differences in electrical conductivity, impedivity, permittivity that can be indicative of pathological tissues.

### EXAMPLE 8

### Strain Elastography and Shear Wave Elastography

In another embodiment, the multi-sensor palpation probe is a robotic finger, and it is adapted to perform either traditional ultrasound (US) imaging or US-based strain elastography (SE) or shear wave elastography (SWE) to evaluate the tissue of interest (FIGS. 45A-45B). The finger (see FIG. IIB) combines multiple sensing modalities to assess comprehensive tissue properties. Mechanical properties are evaluated by compressing the tissue, with a force sensor and elastography imaging providing additional deformation data. An integrated accelerometer delivers real-time information on the finger's position. Electrical properties are measured using an array of contact electrodes. A central microcontroller coordinates data acquisition and communication among all sensing modules. Specifically, the US-based elastography can monitor tissue deformation under applied force (for the strain elastography) or shear wave propagation through the tissue (for the shear wave elastography), allowing for quantifiable tissue structure and integrity. Combination of the US-based elastography and the mechano-electrical tissue assessments can provide a more comprehensive evaluation of tissue structure and mechanical properties and improves the detection and characterization of pathological tissue, such as tumors. Additionally, an imaging module, such as an optical fiber, is integrated in the probe that can provide visual information during probe navigation and tissue assessments.

### EXAMPLE 9

### Strain Elastography and Shear Wave Elastography

In another embodiment, a wirelessly operated multi-modal sensing device combined with mechano-electrical sensors and a US-based elastography device, can be deployed using existing laparoscopic and robotic tools (FIG. 46). In this device configuration, the deployable sensor can be mounted onto robotic arms and the effector end of laparoscopic instruments—such as graspers or surgical manipulators—to enable precise positioning and dynamic control using existing controllers for local tissue assessments. The deployable probe is designed to wirelessly communicate via Bluetooth or Wi-Fi with the data acquisition and processing module for real-time data transmission and analysis (i.e., real-time intraoperative tissue measurement). The probe features wireless (Bluetooth/Wi-Fi) communication with a data acquisition and processing module. This integration enhances adaptability of the probe across diverse surgical environments.

### EXAMPLE 10

### AI-Assisted Tissue Quality Evaluation

A further enhancement of the robotic system incorporates artificial intelligence (AI) algorithms that analyze the combined datasets obtained from the multi-modal mechanical, electrical, and elastography, and optical imaging sensors (FIG. 47). Data from various sensor modalities, including mechanical sensing, electrical sensing, elastography, and imaging, are integrated into a unified data fusion and preprocessing module. The preprocessed, fused data is then analyzed by advanced artificial intelligence (AI) algorithm using machine learning or deep learning, which generate decision support outputs for the device for detection of pathological tissues. US: Ultrasound; SE: strain elastography; SWE: shear wave elastography. ML: machine learning. DL: deep learning. The AI algorithms can be trained to evaluate the biophysical properties of the tissue of interest, identify abnormal patterns, and identify potential disease indicators. By integrating multi-modal data, the AI-assisted system can provide a robust, automated, and reliable evaluation of tissue health, improve diagnostic accuracy, and potentially offer decision support for clinical interventions.

Additional details relating to the present invention are presented in the publication by Jiawen Chen et al. entitled “Non-destructive vacuum-assisted measurement of lung elastic modulus,” Acta Biomaterialia. 2021 September 1;131:370-380, the entire disclosure which is attached incorporated herein by reference and made part of the present disclosure for all purposes. Further additional information is contained in the paper entitled “A Minimally Invasive Robotic Tissue Palpitation Device” by Mohammad Mir et al., IEEE Transactions on Biomedical Engineering, 2024, which is incorporated herein by reference and made part of the present disclosure for all purposes.

It will be understood that the embodiments described herein are merely exemplary and that a person skilled in the art may make many variations and modifications without departing from the spirit and scope of the invention. All such variations and modifications are intended to be included within the scope of the invention.

What is claimed is:

1. A device for measuring properties of a target tissue, said device comprising:
a catheter;
a deployable sensing probe mounted on said catheter and adapted to compress tissue for stiffness measurement;
a motion control module, operatively coupled to said deployable sensing probe and adapted to enable multi-directional device movements of said deployable sensing probe; and
a micro-optical imaging module integrated into said deployable sensing probe.

2. The device of claim 1, wherein said motion control module is configured to effect linear displacement, rotation, and deflection of said deployable sensor probe.

3. The device of claim 1, wherein said deployable sensing probe comprises a force sensor, one or more contact electrodes, and a rigid compression head.

4. The device of claim 3, wherein said force sensor is thin-film based.

5. The device of claim 3, wherein said deployable sensing probe forms part of a force-to-voltage circuit.

6. The device of claim 5, wherein said deployable sensing probe is configured to apply a force to the tissue of interest to generate an electrical signal and associated deformation length, thereby determining bioelectric properties of the tissue of interest.

7. The device of claim 6, wherein said deployable sensing probe is adapted to determine conductivity, impedivity, and permittivity of the tissue of interest.

8. The device of claim 3, wherein said rigid compression head is hemispheric and made of acrylic plastic.

9. The device of claim 3, wherein said one or more contact electrodes comprise pogo pins.

10. The device of claim 1, wherein said catheter is deflectable.

11. The device of claim 10, wherein said motion control module includes one or more servo motors adapted to enable linear movement to said catheter.

12. The device of claim 10, wherein said motion control module includes a plurality of disks and driving wires configured to enable deflection movement of said catheter.

13. The device of claim 10, wherein said motion control module includes a linear servo adapted to enable rotational movement to said catheter.

14. The device of claim 10, wherein said catheter comprises a wire-driven continuum robot constructed with driving disks, driving wires, and flexible tubing.

15. The device of claim 1, wherein said micro-optical imaging module comprises an LED illumination light source, an optical-fiber imaging bundle with an embedded micro-lens, a monochrome CMOS, an achromatic doublet, an objective lens, a filter holder, a fiber bundle adapter, a translating lens mount, and a plurality of extension tubes.

16. The device of claim 15, wherein said optical-fiber imaging bundle is bifurcated, thereby enabling simultaneous imaging and illumination.

17. The device of claim 1, wherein said catheter comprises an imaging channel, and said micro-optical imaging module is adapted for introduction into said imaging channel of said catheter.

18. The device of claim 1, wherein said micro-optical imaging module is adapted for both tissue-and cellular-level imaging.

19. The device of claim 1, further comprising an artificial intelligence module adapted to evaluate quality of the tissue of interest and to detect diseased tissue.

20. The device of claim 19, wherein said artificial intelligence module is adapted to determine an elastic modulus of the tissue of interest.

21. The device of claim 19, wherein said artificial intelligence module is adapted to determine tissue health, compositions, or integrity of the tissue of interest using data obtained via said micro-optical imaging module.

22. The device of claim 1, wherein said deployable sensing probe is adapted to use ultrasound-based elastography.

23. The device of claim 1, wherein said deployable sensing probe is a multi-sensor palpation probe including a robotic finger.

24. The device of claim 23, wherein said robotic finger is adapted to use traditional ultrasound to evaluate the tissue of interest.

25. The device of claim 23, wherein said robotic finger is adapted to use US-based strain elastography to evaluate the tissue of interest.

26. The device of claim 23, wherein said robotic finger is adapted to use shear wave elastography (SWE) to evaluate the tissue of interest.

27. A method for using the device of claim 1, comprising the steps of:
providing a pressure head on said deployable sensor probe;
positioning said deployable sensor probe such that said pressure head is proximate the tissue of interest;
applying a pressure to said pressure head;
detecting a response at the tissue of interest in response to said pressure applied via said applying step; and
calculating one or more physical properties of the tissue of interest based on said response.
